(19) United States
(12) Patent Application Publication
(10) Pub. No.: US 2006/0188958 A1
(43) Pub. Date: Aug. 24, 2006

(54) Artificial enzyme and method for producing the same

(75) Inventors: Tsuyoshi Fujihara, Kawasaki (JP); Michihiko Aki, Kawasaki (JP); Shozo Fujita, Kawasaki (JP)

Correspondence Address: ARMSTRONG, KRATZ, QUINTOS, HANSON & BROOKS, LLP, 1725 K STREET, NW, SUITE 1000, WASHINGTON, DC, 20006, US

(73) Assignee: FUJITSU LIMITED, Kawasaki (JP)

(21) Appl. No.: 11/284,444

(22) Filed: Nov. 22, 2005

(30) Foreign Application Priority Data
Feb. 22, 2005 (JP) 2005-46320

(51) Int. Cl. C07H 21/04; C12P 21/06; C12N 9/00

(52) U.S. Cl. 435/69.1; 435/183; 435/188.5; 435/320.1; 435/325; 536/23.2

(57) ABSTRACT

The method for producing an artificial enzyme according to the invention is a method for producing an artificial enzyme and includes the step of selecting an artificial enzyme precursor which includes an oligonucleotide sequence containing modified nucleosides prepared by introducing a substituent into each nucleoside and at least one of the modified nucleoside capable of reacting with a raw substance of a target reaction which the artificial enzyme catalyzes; and the step of producing the artificial enzyme which is capable of catalyzing a target reaction and includes the oligonucleotide sequence in which the modified nucleoside capable of reacting with the raw substance of the artificial enzyme precursor is substituted with a non-reactive modified nucleoside which is non-reactive with the raw substance of the target reaction.

### CROSS-REFERENCE TO RELATED APPLICATIONS

This application is based upon and claims the benefits of the priority from the prior Japanese Patent Application No. 2005-046320, filed on Feb. 22; 2005, the entire contents of which are incorporated herein by reference.

### BACKGROUND OF THE INVENTION

1. Field of the Invention

The present invention relates to a method for producing an artificial enzyme that can produce easily and efficiently an artificial enzyme which exhibits a desired enzyme activity (catalytic activity) to a desired target reaction without limitation to the type of the target reaction which the artificial enzyme catalyzes and which can be copied. The present invention also relates to an artificial enzyme which is produced by the method for producing an artificial enzyme; of which enzyme activity to a desired target reaction can be controlled; which is easily copied or amplified, recovered, and activated; which can be mass-produced; which allows easy screening of enzyme activity to the target reaction; and which is excellent in handleability.

2. Description of the Related Art

In recent years, a technique has been reported in which molecules having an affinity to a specific target to function as an antibody, molecules functioning as an enzyme which catalyzes a specific reaction, and the like can be copied or amplified easily and efficiently by selecting molecules having a specific function from nucleotide random sequences and identifying them. As one example of such technique, a method is known for selecting molecules having an affinity to a specific target by SELEX method (Systematic Evolition of Ligands by EXponential enrichment). The method is a method for identifying a product from a product library, comprising preparing a mixture of chemically modified nucleic acids, reacting the mixture with a free raw substance of reaction to form the product library, partitioning between members of the product library based on their ability to perform a preselected function (See Japanese Patent Application Laid-Open (JP-A) No. 10-508465). As another example, a method is known for selecting molecules capable of functioning as ribozymes by selecting from a random nucleotide sequence, molecules having catalytic activity under the presence of an effector, of which catalytic activity is regulated by the interaction with the effector (See Japanese Patent Application Laid-Open UP-A) No. 2004-515219). However, in these cases, since both RNA and DNA is composed of 4 nucleotides, the kind of the functional groups present in the random sequence of the nucleotides is significantly limited, thus causing a serious problem that inevitably, both of the type and function of the resulting molecule is remarkably limited. Especially, in the case of the ribozyme, there is a problem in that the ribozyme is very unstable because the molecule constituting it is RNA. Although an attempt to use DNA, more stable than RNA, as a component of a molecule exhibiting enzyme activity similar to the ribozyme is considered; however, it has not succeeded yet.

Under such situations, an attempt has been made to use a modified nucleotide in which a substituent is introduced into the nucleotide in order to increase the kind of the functional groups present in the random sequence of the nucleotides and to give molecules diversity.

In the case where the modified nucleotide is used; however, it is required to remove a naturally-occurring nucleotide corresponding to the modified nucleotide from an experimental system in order to determine that a molecule, in which the modified nucleotide is selectively introduced at the specific site of the random sequence is the molecule to be selected. Removing the naturally-occurring nucleotide from the experimental system, eventually, the kind of the nucleotide is 4+1−1, remaining to be 4, which does not provide a radical solution.

Also, a study has been made to select a molecule capable of functioning as an antibody or enzyme using amino acids or artificial materials as a compositional unit by combinatorial chemistry. However, in this case, it is difficult to determine the structure of the molecule selected finally since the molecule is not composed of the nucleotide which can be copied or amplified. Besides, there is a problem in that the molecule is not conveniently handled because it is not easy to copy or amplify the molecule.

On the other hand, a supramolecule assembly which is coated with virus having a self-replicating function, etc. has been proposed (See JP-A No. 10-508304). However, in this case, there is a problem in that the supramolecule assembly has a complex structure, determination of the structure is not easily, and it is difficult to produce one having an excellent enzyme activity efficiently.

Therefore, a method for producing an artificial enzyme that can produce easily and efficiently an artificial enzyme which exhibits a desired enzyme activity (catalytic activity) to a desired target reaction without limitation to the type of the target reaction which the artificial enzyme catalyzes and which can be copied has not been provided yet. Also, an artificial enzyme which is produced by the method for producing an artificial enzyme; of which enzyme activity to a desired target reaction can be controlled; which is easily copied or amplified, selectively recovered, and activated; which can be mass-produced; which allows easy screening of enzyme activity to the target reaction; and which is excellent in stability, safety, and handleability has not been provided yet.

An object of the present invention is to solve conventional problems and to provide a method for producing an artificial enzyme that can produce easily and efficiently an artificial enzyme which exhibits a desired enzyme activity (catalytic activity) to a desired target reaction without limitation to the type of the target reaction which the artificial enzyme catalyzes and which can be copied; and an artificial enzyme which is produced by the method for producing an artificial enzyme; of which enzyme activity to a desired target reaction can be controlled, which is easily copied or amplified, selectively recovered, and activated, which can be mass-produced, which allows easy screening of enzyme activity to the target reaction, and which is excellent in stability, safety, and handleability.

### SUMMARY OF THE INVENTION

The method for producing an artificial enzyme according to the invention is a method for producing an artificial enzyme comprises the step of selecting an artificial enzyme precursor which comprises an oligonucleotide sequence containing modified nucleosides prepared by introducing a substituent into each nucleoside and at least one of the modified nucleoside capable of reacting with a raw substance of a target reaction which the artificial enzyme catalyzes; and the step of producing the artificial enzyme which is capable of catalyzing the target reaction and comprises the oligonucleotide sequence in which the modified nucleoside capable of reacting with the raw substance of the artificial enzyme precursor is substituted with a non-reactive modified nucleoside which is non-reactive with the raw substance of the target reaction.

According to the method for producing an artificial enzyme of the invention, in the step of selecting an artificial enzyme precursor, the artificial enzyme precursor is selected which comprises an oligonucleotide sequence containing modified nucleosides prepared by introducing a substituent into each nucleoside and at least one of the modified nucleoside capable of reacting with a raw substance of a target reaction which the artificial enzyme catalyzes. In the step of selecting an artificial enzyme precursor, the artificial enzyme precursor comprising the reactive modified nucleoside is selected, and the artificial enzyme precursor can react with the raw substance of the target reaction which the artificial enzyme catalyzes since the artificial enzyme precursor comprises the reactive modified nucleoside and has enzyme activity (catalytic activity) to the target reaction. In the step of producing the artificial enzyme, the artificial enzyme is produced which is capable of catalyzing the target reaction and comprises the oligonucleotide sequence in which the reactive modified nucleoside of the artificial enzyme precursor is substituted with a non-reactive modified nucleoside which is non-reactive with the raw substance of reaction.

The artificial enzyme produced in the artificial enzyme producing step has enzyme activity (catalytic activity) to the target reaction, like the artificial enzyme precursor, but compared to the artificial enzyme precursor, does not comprise the reactive modified nucleoside in the oligonucleotide sequence, instead comprises the non-reactive modified nucleoside. Thus, the artificial enzyme has a simple structure, in addition, is easily copied or amplified, can be mass-produced, and has a self-replicating ability. Because the artificial enzyme comprises the oligonucleotide sequence containing at least one modified nucleoside prepared by introducing a substituent into a nucleoside and has larger force of interaction with other molecules, or the like than the oligonucleotide sequence composed of normal 4 kinds of nucleosides into which the substituent is not introduced, the artificial enzyme has high affinity to (specific reactivity with) the raw substance of reaction, etc. In addition, the artificial enzyme is excellent in stability and safety, is easily recovered using nucleic acids, and has excellent handleability.

In the method for producing an artificial enzyme according to the invention, in one aspect, in the step of selecting an artificial enzyme precursor, the artificial enzyme precursor is preferably selected from a random artificial enzyme precursor pool which comprises two or more kinds of the artificial enzyme precursors, each containing a different oligonucleotide sequence. One advantage of this aspect is that an artificial enzyme exhibiting a desired enzyme activity can be obtained among these selected artificial enzyme precursors because two or more kinds of the artificial enzyme precursors are selected from the random artificial enzyme precursor pool.

In another aspect, preferably, the method further comprises, after the step of selecting the artificial enzyme precursor and before the step of producing the artificial enzyme, a step of sequencing the oligonucleotide sequence of the artificial enzyme precursor selected in the step of selecting the artificial enzyme precursor. One advantage of this aspect is that the place of the reactive modified nucleoside present in the artificial enzyme precursor can be specified.

In another aspect, preferably, in the step of selecting the artificial enzyme precursor, two or more kinds of artificial enzyme precursors are selected. One advantage of this aspect is that an artificial enzyme exhibiting a desired enzyme activity can be easily obtained because the selected two or more kinds of the artificial enzyme precursors normally have different enzyme activity each other.

In another aspect, preferably, the method further comprises, after the step of selecting the artificial enzyme precursor and before the step of producing the artificial enzyme, a step of producing the artificial enzyme precursor which comprises the oligonucleotide sequence sequenced in the step of sequencing the oligonucleotide sequence; and a step of sorting the artificial enzyme precursor having high reactivity with the raw substance of reaction from the artificial enzyme precursor produced by the step of producing an artificial enzyme precursor. One advantage of this aspect is that an artificial enzyme exhibiting a desired enzyme activity can be easily obtained.

In another aspect, preferably, the raw substance of reaction comprises a capture site capable of being captured by a capturing unit, and the selection in the step of selecting an artificial enzyme precursor is carried out by allowing the raw substance of reaction and the reactive modified nucleoside, and then by capturing the capture site of the raw substance of reaction by the capturing unit to thereby capture the reactive modified nucleoside reacted with the raw substance of reaction. One advantage of this aspect is that an artificial enzyme exhibiting a desired enzyme activity can be easily obtained since the selection of the artificial enzyme precursor is efficiently carried out using the capturing unit.

In another aspect, the reactive modified nucleoside in the oligonucleotide sequence of the artificial enzyme precursor is preferably positioned at a portion other than terminus of the oligonucleotide sequence. This aspect enables the active center of an enzyme to be positioned at the center portion of the molecule of the artificial enzyme, which was difficult according to a conventional technique, and is advantageous in that the enzyme activity of the artificial enzyme can be significantly improved.

In another aspect, a reaction between the reactive modified nucleoside and the raw substance of reaction is preferably at least one selected from the group consisting of a Diels-Alder reaction, an amide condensation reaction, an amide bonding reaction. One advantage of this aspect is that the obtained artificial enzyme can be suitably used as an enzyme which catalyzes each of the reactions.

In another aspect, the substituent is preferably selected from a group represented by one of the following Structural Formulae (I) and (I′). One advantage of this aspect is that a desired affinity to the raw substance of reaction can be provided with the artificial enzyme and the artificial enzyme can exhibit a desired enzyme activity (catalytic activity). 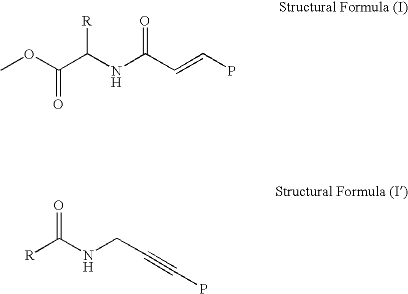
where R represents any one selected from a natural or nonnatural amino acid, a metal complex, a fluorescent dye, a oxidation-reduction pigment, a spin label compound, a hydrogen atom, an alkyl group having carbon number ranging from 1 to 10, and groups represented by the following formulae (1) to (16); and P represents the pyrimidine base.

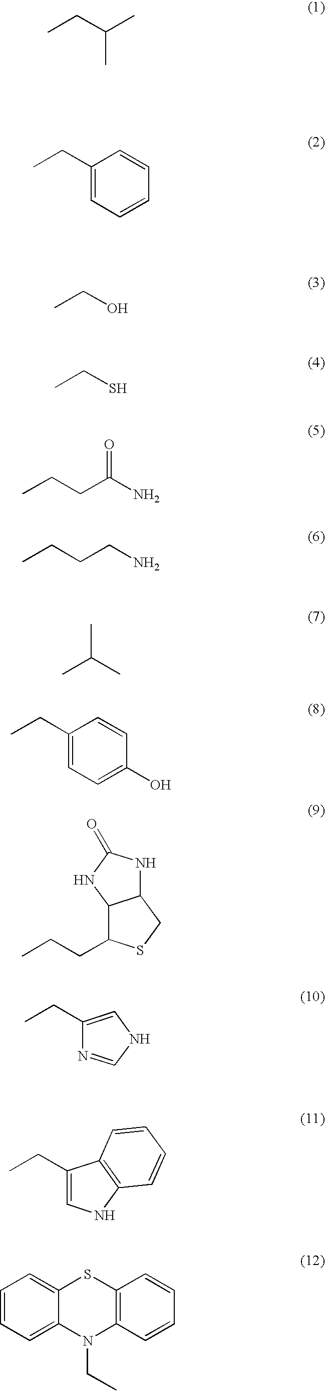

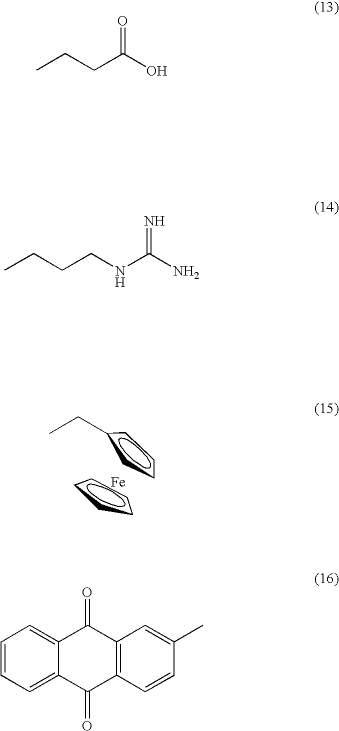

The artificial enzyme according to the invention exhibits enzyme activity to a target reaction and is produced by the method for producing an artificial enzyme of the invention.

Since the artificial enzyme according to the invention comprises the oligonucleotide sequence, it has a self-replicating ability, is easily copied or amplified, can be mass-produced, and is excellent in stability. Further, since the artificial enzyme comprises the oligonucleotide sequence, it can be easily and selectively recovered by hybridization, etc. using nucleic acids, and since the hybridized artificial enzyme can be reused by thermally melting it by heating, the artificial enzyme according to the invention has excellent handleability. Further, the artificial enzyme can be easily obtained as a molecule having enzyme activity (catalytic activity) to a desired reaction by a certain method (method for producing an artificial enzyme according to the invention) and has excellent versatility. Further, since the artificial enzyme comprises the oligonucleotide sequence, it can, for example, be linked to an antibody formed using nucleic acids and can be suitably used for designing a multifunctional molecule having both an antibody function and enzyme function. Further, since the artificial enzyme is formed of biomolecules, it is excellent in safety and can be suitably used in a variety of fields, including pharmaceuticals, drug deliveries, and biosensors.

### BRIEF DESCRIPTION OF THE DRAWINGS

FIG. 1 is a schematic view illustrating an example of a DNA synthesizer used for synthesizing a random polymer pool.

FIGS. 2A to 2E are schematic explanatory views illustrating an example of the artificial enzyme precursor selecting step in the method for producing an artificial enzyme, in the case of the artificial enzyme precursor catalyzing a bonding reaction.

FIGS. 3A to 3D are schematic explanatory views illustrating an example of the artificial enzyme precursor selecting step in the method for producing an artificial enzyme, in the case of the artificial enzyme precursor catalyzing a breakdown reaction.

FIG. 4A is a schematic view explaining a status, where the random polymer pool is synthesized, in the method for producing an artificial enzyme according to the invention; FIG. 4B is a schematic view illustrating an example of the selection of an artificial enzyme precursor by an affinity column; FIG. 4C is a schematic view explaining a state, where the base sequence of an oligonucleotide sequence after the selection and recovery of the oligonucleotide sequence is being determined; and FIG. 4D is a schematic view explaining a state, where the base sequence of the oligonucleotide sequence is translated.

### DESCRIPTION OF THE PREFERRED EMBODIMENTS

(Artificial Enzyme and Method for Producing the Same)

The method for producing an artificial enzyme according to the invention is a method for producing an artificial enzyme, comprises an artificial enzyme precursor selecting step and an artificial enzyme producing step, and may comprise other steps such as a sorting step, oligonucleotide sequencing step, and translating step.

The artificial enzyme according to the invention is produced by the method for producing an artificial enzyme of the invention.

The method for producing an artificial enzyme according to the invention will be described in detail below, and besides, through the description, the artificial enzyme according to the invention will be explained in detail.

—Artificial Enzyme Precursor Selecting Step—

The artificial enzyme precursor selecting step is a step in which an artificial enzyme precursor is selected which comprises an oligonucleotide sequence containing modified nucleosides prepared by introducing a substituent into each nucleoside and at least one of the modified nucleoside capable of reacting with a raw substance of a target reaction which the artificial enzyme catalyzes.

<Artificial Enzyme Precursor >

The artificial enzyme precursor comprises the oligonucleotide sequence, and the oligonucleotide sequence is formed of the modified nucleoside and comprises at least one reactive modified nucleoside. Among the artificial enzyme precursors, those comprising an oligonucleotide sequence in which a nucleotide n-mer (where, n represents an integer) containing the modified nucleoside is randomly polymerized are preferable in terms of easy synthesis, etc.

—Modified Nucleoside—

The modified nucleoside is the nucleoside into which the substituent is introduced. The modified nucleoside is not a naturally-occurring nucleoside (adenosine, guanosine, thymidine, cytidine), but a nucleic acid derivative.

—Nucleoside—

The nucleoside is a molecule constituting nucleic acids and is preferably one constituting at least DNA and RNA from the viewpoint of molecular design and the like.

Of the nucleoside, a deoxynucleoside constitutes the DNA. Specifically, examples of the deoxynucleoside include deoxyadenosine (dA), deoxyguanosine (dG), deoxycytidine (dC) and thymidine (T), which correspond to 4 kinds of bases constituting the DNA, i.e., adenine (A), thymine (T), guanine (G) and cytosine (C).

Of the nucleoside, a ribonucleoside constitutes the RNA. Examples of the ribonucleoside include adenosine (A), guanosine (G), cytidine (C) and uridine (U), which correspond to 4 kinds of bases constituting the RNA, i.e., adenine (A), guanine (G), uracil (U) and cytosine (C).

The nucleoside is be used alone or two or more may be used in combination.

—Substituent—

The substituent is not particularly limited and may be properly selected depending on the application. For example, a group represented by one of the following Structural Formulae (I) and (I′) or the like is preferred in that it is easily synthesized or it has diverse quality itself.

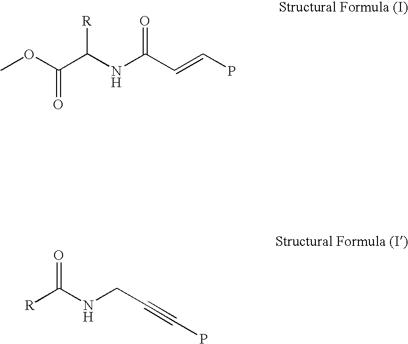

In the Structural Formulae (I) and (I′), R represents any one selected from a natural or nonnatural (synthetic) amino acid, a metal complex, a fluorescent dye, a oxidation-reduction pigment, a spin label compound, a hydrogen atom, an alkyl group having carbon number ranging from 1 to 10, and groups represented by the following formulae (1) to (16). P represents the pyrimidine base.

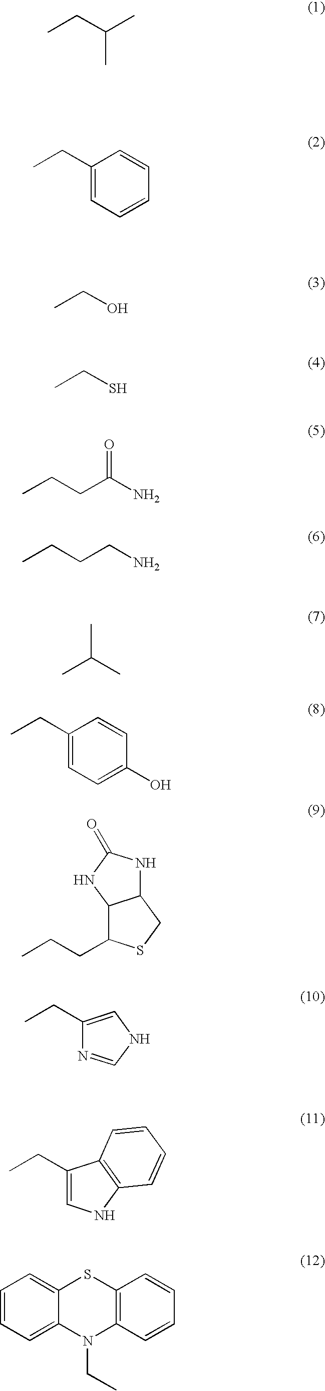

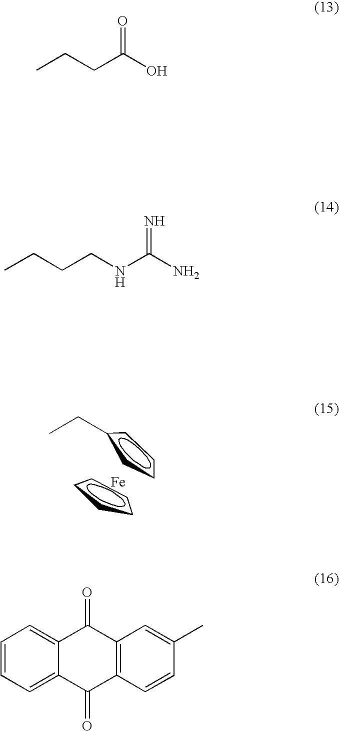

The amino acid is not particularly limited, may be properly selected depending on the application and may be natural amino acid or synthesized amino acid. Examples of the amino acid include 20 kinds of amino acids making up proteins.

The metal complex is not particularly limited and may be properly selected depending on the application as long as it is a compound in which a ligand is coordinated with a metal ion, including, for example, Ru-bipyridil complex, ferrocene complex, nickel imidazole complex, and the like.

The fluorescent dyes are not particularly limited, and may be properly selected depending on the application, including, for example, fluorescent dyes such as fluorescein dyes, rhodamine dyes, eosine dyes, NBD dyes, and the like.

The oxidation-reduction pigments are not particularly limited, and may be properly selected depending on the application, including, for example, leuco pigments such as leucoaniline, leucoanthocyanine, and the like.

The spin label compounds are not particularly limited, and may be properly selected depending on the application, including, for example, iron N-(dithiocarboxy) sarcosine, TEMPO (tetramethyl piperidine) derivatives, and the like.

The alkyl groups having carbon number ranging from 1 to 10 are not particularly limited, and may be properly selected depending on the application, including, for example, a methyl group, ethyl group, propyl group, isopropyl group, butyl group, isobutyl group, tert-butyl group, pentyl group, neopentyl group, hexyl group, cyclohexyl group, octyl group, nonyl group, decyl group and the like.

The substituent may be further substituted by a known substituent. Further, the substituent may be introduced (included) independently or two or more of substituents may be introduced (included).

The enzyme activity, ability of recognizing the reaction object (affinity to the reaction object), and the like of the artificial enzyme according to the invention may be desirably adjusted by appropriately changing or adjusting the number of the substituents to be introduced into the nucleoside, the place where the substituent is to be introduced, the type of the substituent.

In the invention, of the above-mentioned substituents, groups represented by the Formulae (1) to (16) are preferable in that the enzyme activity of the artificial enzyme, ability of recognizing the reaction object (affinity to the reaction object), and the like of the artificial enzyme is easily controlled or adjusted.

The position of the substituent in the modified nucleoside is not particularly limited and may be properly selected depending on the application. Examples of the position include the 5th position in pyrimidine, the 7th position in 7-deazapurine, the 8th position in purine, substitution of an amine outside a ring, substitution of 4-thiouridine, substitution of 5-bromo, substitution of 5-iodouracil, and the like.

Among these, the 5th position in pyrimidine and the 7th position in deazapurine, and the like are preferable in that introduction of the substituent into such position hardly inhibit an enzyme reaction at amplification (replication), and further, the 5th position in pyrimidine is more preferable in terms of easy synthesis.

The substituent may be introduced into the nucleoside by any method without limitation, in other words, the modified nucleoside is synthesized by any method without limitation and the method may be properly selected depending on the application. For example, the substituent is preferably introduced by the method shown in the following formula, in which substituent R is introduced into the 5th position in pyrimidine base of the nucleoside.

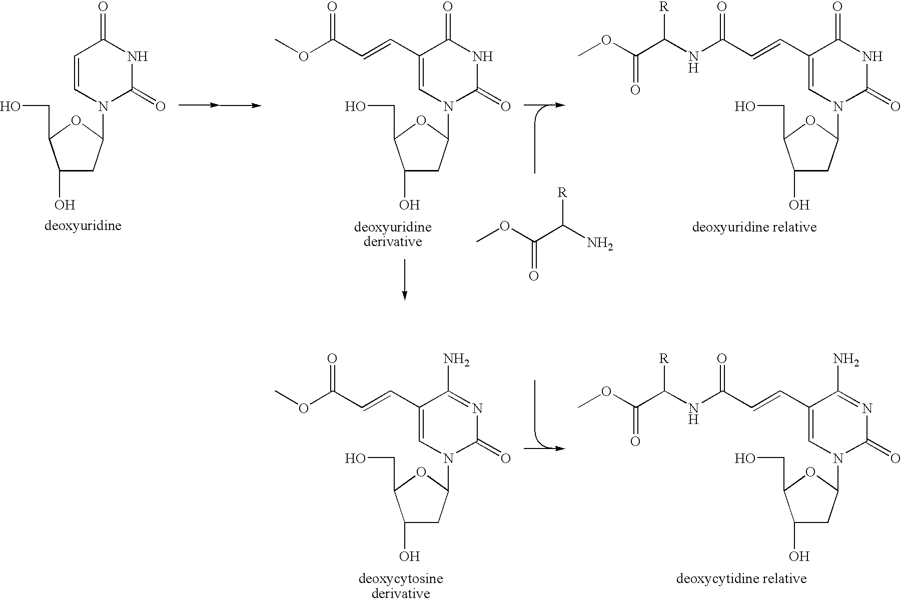

The oligonucleotide sequence comprises the modified nucleoside. Specifically, the sugar part of the modified nucleoside is preferably linked with phosphoric acid through an ester bond, and is more preferably formed of nucleotide n-mer(s) in terms of easy synthesis.

“n” in the nucleotide n-mer represents an integer, is preferably 2 or more, more preferably 2 to 10, particularly preferably 2 to 3.

When the n is less than 2, number of kinds of the nucleotide is not almost different from 4 kinds of nucleotides composing nucleic acids, thus sometimes causing insufficient improvement of the ability of recognizing the raw substance of reaction (affinity to the raw substance of reaction). On the other hand, when the “n” is 4 or more, the load on the synthesis may increase. Specifically, when a single base deletion or single base addition is caused during copying or amplifying the oligonucleotide sequence containing the nucleotide n-mer, oligonucleotides having correct sequence are difficult to be distinguished from those having such deletion or addition. Even if the “n” is 3, up to 64 kinds of different side chains can be introduced. Therefore, considering that various kinds of proteins are made up from 20 kinds of amino acids (valine, leucine, isoleucine, alanine, arginine, glutamine, lysine, aspartic acid, glutamic acid, praline, cysteine, threonine, methionine, histidine, phenylalanine, tyrosine, tryptophan, asparagine, glycine, serine), even if n is 3, it is advantageous in that sufficiently various kinds of molecules can be obtained, and thus n=3 is enough without increasing the load on the synthesis.

Nucleotide dimer, which corresponds to n=2 in the nucleotide n-mer, is not particularly limited and may be properly selected depending on the application. For example, it may be a combination of one of 4 kinds of nucleosides composing nucleic acids and the modified nucleoside or a combination of the modified nucleosides.

The nucleotide dimer may be synthesized by any method without limitation and the method may be properly selected depending on the application. Examples thereof include a diester method, triester method, phosphite method, phosphoramidite method, H-phosphonate method, thiophosphite method, and the like. Among these methods, the phosphoramidite method is preferable.

The phosphoramidite method is, as a rule, employs a condensation reaction of nucleoside phosphoroamidite and the nucleoside as a key reaction using tetrazole as an enhancer. This reaction usually occurs competitively both in a hydroxyl group of a sugar part and an amino group of a base part of the nucleoside. However, in order to realize the desired nucleotide synthesis, the reaction should occur selectively only in the hydroxyl group of the sugar part. Hence, for inhibition of a side reaction to the amino group, the amino group is required to be modified with a protecting group.

Specific example of the method for synthesizing the nucleotide dimer is as follows. As shown by the following formula, nucleotide dimer (AU₁) can be synthesized from deoxyadenosine and modified deoxyuridine.

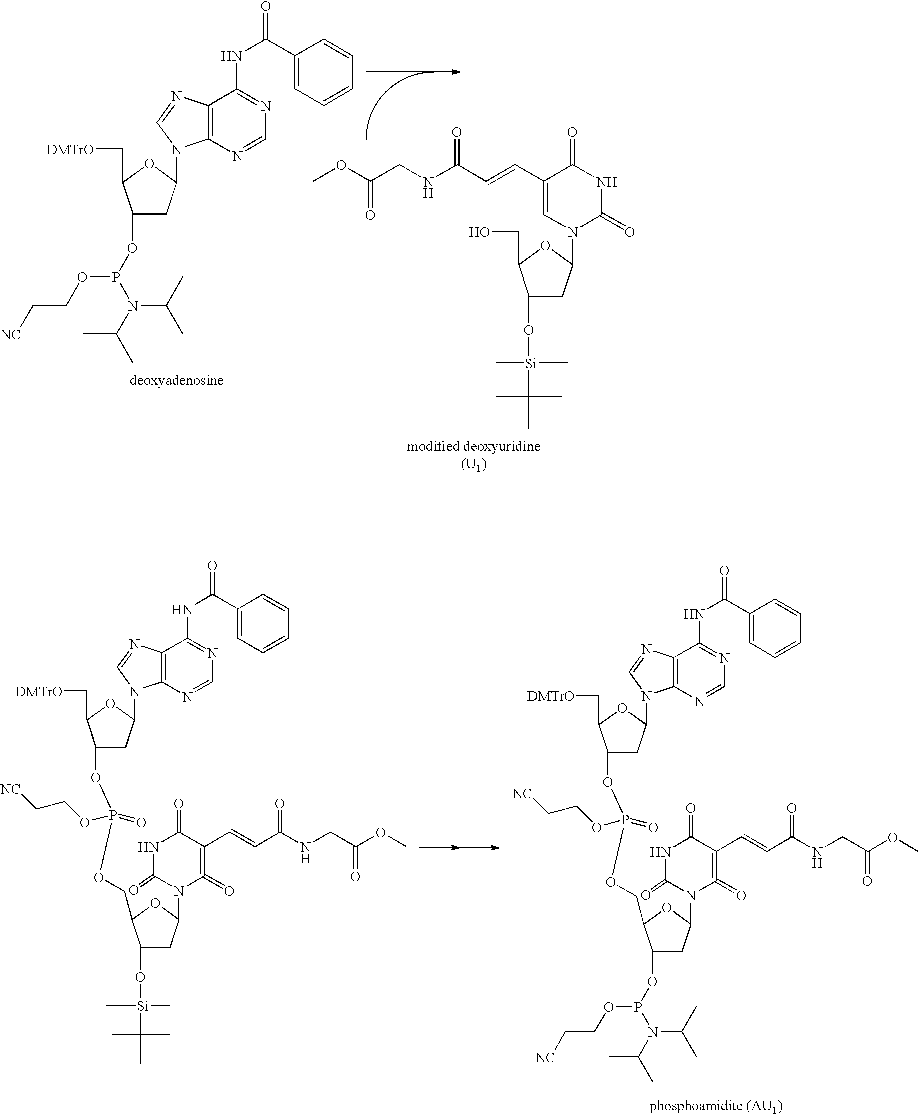

In the formula, DMTr represents a dimethoxytrityl group.

Nucleotide dimers (AC₁, C₂A, C₃C, C₄G, C₅T, GC₆, GU₂, U₃A, U₄C, U₅G, and U₆T), which are shown in the following relation table mentioned later, can be also synthesized by a similar method.

Here, the synthesized nucleotide dimers are made to one-to-one correspond with (relate to) any one of 16 nucleosides in the relation table, which is made by combining 4 kinds of nucleosides constituting nucleic acids, in a one-to-one format.

The number of kinds of the nucleotide dimer is not particularly limited, may be properly selected depending on the application, but is, for example, preferably 5 to 16. When the kind of the nucleotide dimer is less than 5, there is no large difference from 4 kinds of nucleotides constituting the nucleic acid to invite insufficient improvement of the ability of recognizing the reaction object (affinity to the reaction object).

The relation table is, for example, exemplified by that shown in the following Table 1. In this Table 1, bases of 4 kinds of nucleosides are transversely (5′ side) arranged in the order of A, C, G and T and, on the other hand, bases of 4 kinds of nucleosides are longitudinally (3′ side) arranged in the order of A, C, G and T, and 16 patterns (boxes) are formed by relating these bases to each other one-to-one.

TABLE 1

5′

| 3′ | A | C | G | T |
|---|---|---|---|---|
| A | — | C₂A | — | U₃A |
| C | AC₁ | C₃C | GC₆ | U₄C |
| G | — | C₄G | — | U₅G |
| T | AU₁ | C₅T | GU₂ | U₆T |

Similarly to the case of the nucleotide dimer, a nucleotide trimer can be used. The relation table in this case is, for example, exemplified by that shown in the following Table 2. In Table 2, 56 patterns (56 kinds of nucleotide trimer) are formed.

The number of kinds of the nucleotide trimer is not particularly limited, may be properly selected depending on the application, but is, for example, preferably 5 to 56. When the kind of the nucleotide trimer is less than 5, there is no large difference from 4 kinds of nucleotides constituting the nucleic acid to invite insufficient improvement of the ability of recognizing the reaction object (affinity to the reaction object).

Similarly to the case of the nucleotide dimer and the nucleotide trimer, a nucleotide n-mer (n representing the integer) can be used. In the relation table in this case, 4ⁿ patterns (4ⁿ kinds of the nucleotide n-mers) can be made.

The number of kinds of the nucleotide n-mer is not particularly limited, may be properly selected depending on the application, but is, for example, preferably 5 to 4ⁿ. When the kind of the nucleotide n-mer is less than 5, there is no large difference from 4 kinds of nucleotides constituting the nucleic acid to invite insufficient improvement of the ability of recognizing the reaction object (affinity to the reaction object).

As a specific example of the nucleotide n-mer, an oligonucleotide amidite is preferable.

TABLE 2

| | A | | C | | G | | T | |
|---|---|---|---|---|---|---|---|---|
| A | A | G | A | G | A | G | A | G |
| | — | — | C₂AA | C₃AG | — | — | U₃AA | U₄AG |
| | C | T | C | T | C | T | C | T |
| | AAC₁ | AAU₁ | C₄AC | C₅AT | GAC₆ | GAU₂ | U₅AC | U₆AT |
| C | A | G | A | G | A | G | A | G |
| | AC₇A | AC₈G | C₁₁CA | C₁₂CG | GC₁₅A | GC₁₆G | U₇CA | U₈CG |
| | C | T | C | T | C | T | C | T |
| | AC₉C | AC₁₀T | C₁₃CC | C₁₄CT | GC₁₇C | GC₁₈T | U₉CC | U₁₀CT |
| G | A | G | A | G | A | G | A | G |
| | — | — | C₂₀GA | C₂₁GG | — | — | U₁₃GA | U₁₄GG |
| | C | T | C | T | C | T | C | T |
| | AGC₁₉ | AGU₁₁ | C₂₂GA | C₂₃GT | GGC₂₄ | GGU₁₂ | U₁₅GC | U₁₆GT |
| T | A | G | A | G | A | G | A | G |
| | AU₁₇A | AU₁₈G | C₂₅TA | C₂₆TG | GU₂₁A | GU₂₂T | U₂₅TA | U₂₆TG |
| | C | T | C | T | C | T | C | T |
| | AU₁₉C | AU₂₀T | C₂₇TC | C₂₈TT | GU₂₃C | GU₂₄T | U₂₇TC | U₂₈TT |

In the relation table in Table 1, as shown above, 12 kinds of the nucleotide dimers are conditioned. Specifically, a base sequence is read from a 5′ side to a 3′ side directions and a base sequence AC corresponds to the nucleotide dimer AC₁. A sequence AT corresponds to the nucleotide dimer AU₁. A base sequence CA corresponds to the nucleotide dimer C₂A. A base sequence CC corresponds to the nucleotide dimer C₃C. A base sequence CG corresponds to the nucleotide dimer C₄G. A base sequence CT corresponds to the nucleotide dimer C₅T. A base sequence GC corresponds to the nucleotide dimer GC₆. A base sequence GT corresponds to the nucleotide dimer GU₂. A base sequence TA corresponds to the nucleotide dimer U₃A. A base sequence TC corresponds to the nucleotide dimer U₄C. A base sequence TG corresponds to the nucleotide dimer U₅G. A base sequence TT corresponds to the nucleotide dimer U₆T.

Conditioning of a base sequence and the nucleotide dimer in the relation table in Table 1 is not particularly limited, and may be properly selected depending on the application. Table 1 is simply an example. When it is difficult to prepare 12 kinds of the nucleotide dimer, a portion may be duplicated. However, it may result in lowering the ability of recognizing the reaction object (affinity to the reaction object). For AA, AG, GA and GG being combinations of purine bases in the relation table in Table 1, no nucleotide dimer was prepared due to a lower reactivity of an enzyme used for modification with the purine base. This does not mean that the nucleotide dimer containing the purine bases alone cannot be prepared.

On the basis of the relation table in Table 1, making a relation between 12 kinds of nucleoside dimer allows increasing the 4 kinds in the conventional nucleic acid to 12 kinds to enable to express distinguishing ability (affinity) to many kinds of the reaction object.

The number of nucleotides in the oligonucleotide sequence is not particularly limited, may be properly selected depending on the application, but is, for example, preferably 10 to 100, more preferably 10 to approximately 50.

When the number of nucleotides is less than 10, diversity can not be obtained. When the number of nucleotides exceeds 100, it may be substantially impossible to prepare the number of molecules fulfilling diversity.

The oligonucleotide sequence may be composed of only random oligonucleotide sequence which is a random sequence of any nucleotide, or may be composed of the random oligonucleotide sequence and a fixed oligonucleotide sequence which is a fixed sequence consisting of same kinds of nucleotides or having a desired nucleotide sequence.

The oligonucleotide sequence may comprise the fixed oligonucleotide sequence, which is advantageous in that the fixed oligonucleotide sequence can be used as a primer during amplification of a nucleic acid. The number of nucleotides of the fixed oligonucleotide sequence is not particularly limited, may be properly selected depending on the application; usually 15 or more nucleotides are preferable and 20 to about 40 nucleotides are more preferable.

The oligonucleotide sequence can be produced or formed by any method without limitation and the method may be properly selected depending on the application. For example, nucleotide monomers or nucleotide monomer blocks (e.g., the oligonucleotide dimer) are annealed to a nucleotide random sequence, and the nucleotide monomers are linked using at least one of a DNA ligase and an RNA ligase to synthesize the oligonucleotide sequence. However, the oligonucleotide sequence is preferable synthesized using a DNA synthesizer (automated DNA synthesizer) or the like.

The DNA ligase is an enzyme to catalyze formation of a covalent bond between the 5′ phosphate group and the 3′ hydroxyl group of adjacent nucleotides. The RNA ligase is an enzyme to allow 5 a 5′ phosphoryl-terminated polynucleotide and a 3′ hydroxyl-terminated polynucleotide to be linked. The substrate of the RNA ligase is originally RNA, however, the RNA ligase also efficiently links a 5′ phosphoryl-terminated polydeoxyribonucleotide and polydeoxyribonucleotide of which only 3′ terminus is ribonucleotide.

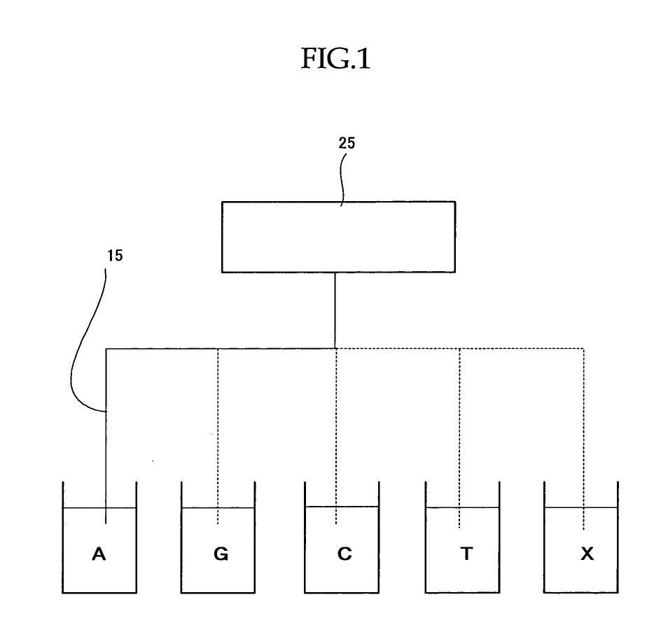

The method using the DNA synthesizer (automated DNA synthesizer) is not particularly limited and may be properly selected depending on the application. For example, the following method or the like is preferable. Specifically, using the DNA synthesizer (automated DNA synthesizer) as shown in FIG. 1, a mixture of a plurality of kinds of the synthesized nucleotide dimer as a reagent (there are 12 kinds in the example shown in FIG. 1; expressed as “X” in FIG. 1) is sucked up by a nozzle 15 and polymerized under control of a controller 25 to thereby prepare the random polymer pool comprising oligonucleotide sequences of which sequences are random and consisting of any sequence order. This method is advantageous in that the random polymer pool is efficiently prepared.

The reactive modified nucleotide which will be mentioned later may be sucked up independently by the nozzle 15 of the DNA synthesizer to be used in polymerization, or may be incorporated in the nucleotide dimer as one of the modified nucleotide units forming the nucleotide dimer and be sucked up by the nozzle 15 of the DNA synthesizer to be used in polymerization.

Preferably, the oligonucleotide sequence comprises at least one reactive modified nucleoside mentioned later and is formed of the modified nucleoside, and may further comprise a monomer or oligomer of DNA or RNA, which is not modified with the substituent according to necessity.

The oligonucleotide sequence produced or formed (synthesized) as mentioned above may be one kind, but is preferably two or more kinds for allowing efficient screening (choice) of an artificial enzyme exhibiting a desired enzyme activity. In the latter case, a random polymer pool comprising a plurality of oligonucleotide sequences can be obtained, and a desired artificial enzyme precursor can be sorted from the random polymer pool. One advantage of this case is that an artificial enzyme exhibiting a desired enzyme activity can be screened efficiently.

Among these oligonucleotide sequences, those having fixed oligonucleotide sequences at both ends are preferable in that amplification of nucleic acids is facilitated.

The fixed oligonucleotide sequence is not particularly limited and may be properly selected depending on the application.

Example thereof include DNA sequences consisting of bases selected from adenine, guanine, cytosine, and thymine; RNA sequences consisting of bases selected from adenine, guanine, cytosine, and uracil; a poly A sequence, a poly T sequence, a poly G sequence, a poly C sequence, a poly U sequence, and the like.

The length of the fixed oligonucleotide sequence is not particularly limited and may be properly selected depending on the application, but is, for example, preferably 4 to 100, more preferably 10 to 50.

The fixed oligonucleotide sequence can be synthesized by any method without limitation and the method may be properly selected depending on the application. For example, similar methods as the method for synthesizing the oligonucleotide are preferable. For the synthesis of the fixed oligonucleotide sequence, predetermined modified nucleotide selected from the nucleotides may be used or 4 kinds of nucleotides, i.e. adenine (A), thymine (T), guanine (G), and cytosine (C), may be used.

As the oligonucleotide sequence, those having antibody activity (affinity or binding properties) to the raw substance of reaction are preferable for catalyzing the reaction of the raw substance of reaction efficiently and improving reaction efficiency.

For improving antibody activity of the oligonucleotide sequence to the raw substance of reaction, for example, structures having antibody activity (affinity or binding properties) to the raw substance of reaction may be introduced into the oligonucleotide sequence. For example, a capturer capable of capturing the raw substance of reaction may be introduced.

The number of the capturers to be introduced into the oligonucleotide sequence, position (site) at which the capturer is introduced, type of the capturer, etc. are not particularly limited and may be properly selected depending on the application.

The number of the capturers to be introduced into the oligonucleotide sequence is not particularly limited, may be properly selected depending on the application, and is, for example, preferably 1 to 2. When the number to be introduced is 2 or more, the 2 or more capturer may be the same as each other or may be different from each other.

The position (site) at which the capturer is introduced into the oligonucleotide sequence is not particularly limited and may be properly selected depending on the application. For example, end (both ends) of the oligonucleotide sequence, and the like are preferable.

The type of the capturer is not particularly limited, is different depending on how the raw substance of reaction is captured, for example, different among adsorption, chemical bonding, and the like, and cannot be clearly defined unconditionally. Examples of the type of the capturer include antibodies, proteins, nucleic acids, parts (segments) of these, and the like. Among these, nucleic acids are more preferable in the they are easily copied or amplified.

Examples of the antibody include a polyclonal antibody, monoclonal antibody, and the like. Examples of the part of antibody include an antibody light chain variable region, antibody heavy chain variable region, antibody (Fab)₂fragment, antibody F(ab′)₂fragment, and the like. These may be used alone or two or more may be used in combination.

The polyclonal antibody usually has affinity for a number of antigenic determinants (epitope). Examples of the polyclonal antibody include an immune antibody resulting from a pathogenic microorganism infection, antiserum, autoantibody, and the like.

The monoclonal antibody has affinity for a single antigenic determinant (epitope). The monoclonal antibody can be produced by, for example, monoclonal antibody producing cell which is formed by cell fusion between sensitized B cells and myeloma cell lines.

The antibody light chain variable region means the 110-amino acid sequence portion from N terminus (variable region) in the two light peptide chain (L chain) having a molecular weight of about 23,000 of immunoglobulin IgG.

The antibody heavy chain variable region means the 110-amino acid sequence portion from N terminus (variable region) in the two heavy peptide chain (H chain) having a molecular weight of 50,000 to 70,000 of immunoglobulin IgG.

The antibody (Fab)₂ fragment is a fragment of immunoglobulin IgG. When the immunoglobulin IgG is digested by papain, it is cleaved into two Fab portions, and one Fc portion which binds to a complement binding site or Fc receptor of cell. The antibody (Fab)₂ fragment means the these two Fab portions binding to antigens.

The antibody F(ab′)₂ fragment means the portion specifically binds to antigens which is obtained as a result of digestion of immunoglobulin IgG by pepsin.

The protein is not particularly limited and may be properly selected from the proteins other than the antibody or part thereof depending on the application. Examples include a peptidoglycan recognition protein (hereinafter may be referred to as “PGRP”) which specifically binds to peptidoglycan (PG) constituting the cell walls of most prokaryotes; a lipopolysaccharide (LPS) binding protein which specifically binds to lipopolysaccharide constituting the outer membrane of gram-negative bacteria (Eur. J. Biochem. Vol. 1248, pp. 217-224, 1997.); a βG recognition protein which specifically binds to βG constituting the cell walls of fungi (The Journal of Biological Chemistry Vol. 263, No 24, pp. 12056-12062, 1988.), and the like.

Examples of the nucleic acids include aptamers, nucleic acids capable of capturing targets, and the like.

The aptamer is a nucleic acid molecule which recognizes small molecules such as amino acids and proteins, and besides, macromolecules such as viruses, can be synthesized or copied in large amount, is easily adjusted, and has a property as a RNA antibody which specifically binds to the target. The aptamer is applicable to function-blocking of cancer-causing factor (cancer suppressor), quantitative measurement of cancer associated factors (cancer diagnosis), development of RNA molecules which mimics bioactive proteins (drug discovery), and the like.

The aptamer is not particularly limited and may be properly selected from the depending on the application. For example, the oligonucleotide sequence mentioned above, and the like are preferable.

Preferably, binding portion (linker), through which the capturer is boned to the oligonucleotide sequence, can be copied or amplified. Examples include nucleic acids, amino acid sequences, polymer chains, complexes thereof, and the like.

When the binding portion is the nucleic acid, the nucleic acid may include a hairpin structure. When the hairpin structure is formed at the binding portion, it gives convenience during sorting, structure decision, or the like, and advantageous in that cooperative effect may be also expected.

—Reactive Modified Nucleoside—

The reactive modified nucleoside is not particularly limited as long as it exhibits reactivity or can react with the raw substance of the target reaction which the artificial enzyme catalyze (exhibits enzyme activity), and may be properly selected depending on the application. For example, those in which a structure exhibiting reactivity with the reaction object of the reaction which the artificial enzyme catalyzes is introduced into part of the above-mentioned modified nucleoside are exemplified. In this case, the reactive modified nucleoside is different from the modified nucleoside in that although the former exhibits reactivity with the raw substance of reaction, the latter does not.

The reactive modified nucleoside is not particularly limited and may be properly selected depending on the application. For example, preferably, the reactive modified nucleoside is at least one selected from uridine, cytidine, 7-deazaadenine, 7-deazaguanine, and is a derivative thereof, and a uridine derivative having a structure capable of reacting with the raw substance of reaction in a portion thereof, and the like are more preferable.

The target reaction is a similar reaction as the reaction which is a target which the artificial enzyme catalyzes, is not particularly limited and may be properly selected depending on the application. For example, a bonding reaction, breakdown reaction, and the like are preferable. By the reaction of the reactive modified nucleoside and the reaction object, the reactivity of the artificial enzyme precursor with or affinity of the artificial enzyme precursor to the reaction object can be judged. Thus, the artificial enzyme precursor exhibiting reactivity with or affinity to the reaction object is selected from a number of various artificial enzyme precursors, and the reactive modified nucleoside of the artificial enzyme precursor is replaced by a non-reactive nucleoside which does not exhibit reactivity with the reaction object to thereby obtain the artificial enzyme according to the invention.

Examples of the bonding reaction include a polymerization reaction, condensation reaction, condensation polymerization reaction, addition reaction, polyaddition reaction, and the like.

The chemical bond involving in the bonding reaction is not particularly limited and may be properly selected depending on the application. Examples thereof include a covalent bond, coordination bond, ion bond, hydrogen bond, and the like. As a specific example of the bonding reaction, a Diels-Alder reaction, amide condensation reaction, amide bonding reaction, ester bonding reaction, and the like are preferable in terms of stability of products.

Examples of the breakdown reaction (cleavage reaction) include a hydrolysis reaction, a cutting by a substitution reaction, and the like.

The bond to be broken down is not particularly limited and may be properly selected depending on the application. Examples of the bond to be broken down include an ester bond, amide bond, and the like.

Here, the Diels-Alder reaction, which was mentioned as one of the specific examples of the bonding reaction, will be described. The Diels-Alder reaction is a ring forming reaction represented by the following reaction formula. Namely, two compounds shown on the left side of the reaction formula are raw substance of reactions of the Diels-Alder reaction, and the two raw substance of reactions react with each other selectively to form a ring.

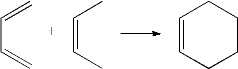

The raw substance of reaction is not particularly limited and may be properly selected according to the type of the target reaction, or the like. For example, in the case where the target reaction is the Diels-Alder reaction, a compound having in a portion thereof one of the structure represented by the following Formula A and the structure represented by the following Formula B.

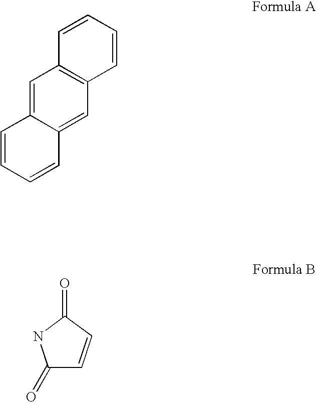

When the raw substance of reaction is, for example, a compound having the structure represented by the Formula B at least in a portion thereof, a compound having the structure represented by the Formula C and the like are preferable as the reactive modified nucleoside.

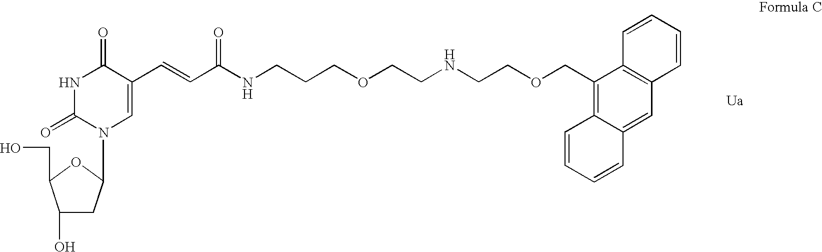

When the reactive modified nucleoside is a compound having the structure represented by the Formula C, for example, a compound having the structure represented by the following Formula D and the like can be suitably used for selection, and can be suitably used as a selecting unit mentioned later. The selecting unit is a unit for selecting the artificial enzyme precursor into which the reactive modified nucleoside is introduced from a number of various oligonucleotide sequences.

The compound having the structure represented by the Formula D has an imide group at one end, which is capable of reacting with the anthracene structure contained in the structure represented by the Formula C through a Diels-Alder reaction. In addition, since the compound having the structure represented by the Formula D has a biotin structure which allows separation by adsorption using an avidin column, it can be suitably used as the selecting unit.

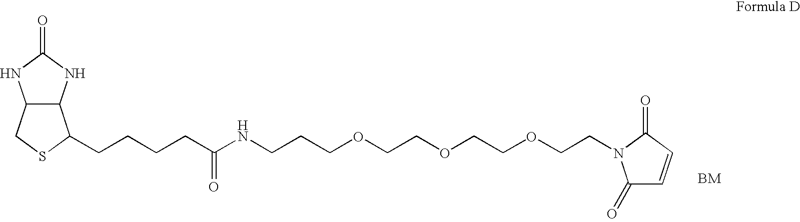

The reaction between the compound having the structure represented by the Formula C and the compound having the structure represented by the Formula D is the Diels-Alder reaction which occurs between the anthracene structure moiety of the compound having the structure represented by the Formula C and the imide group of the compound having the structure represented by the Formula D.

Specifically, the reaction is represented as follows.

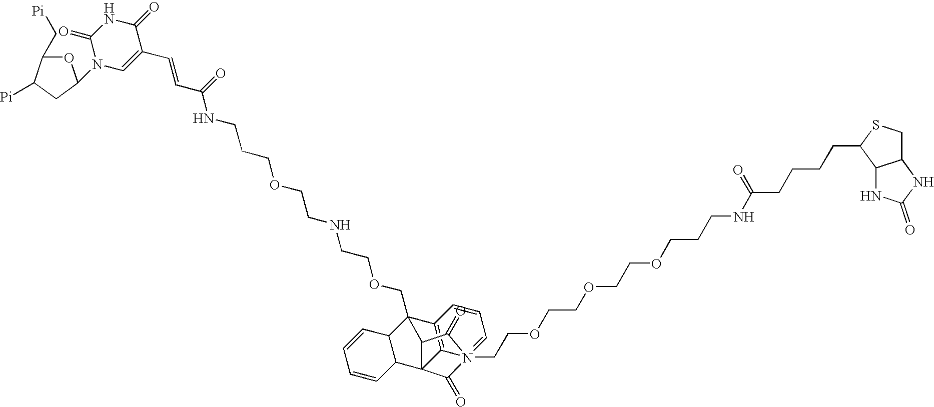

When the reactive modified nucleoside is the compound having the structure represented by the Formula C, the artificial enzyme, in which the reactive modified nucleoside of the artificial enzyme precursor having the reactive modified nucleoside is substituted with a non-reactive modified nucleoside, can catalyze the following Diels-Alder reaction. Namely, the target reaction is the Diels-Alder reaction and the anthracene compound and imide compound which are shown on the left side of the reaction formula are the raw substance of reactions.

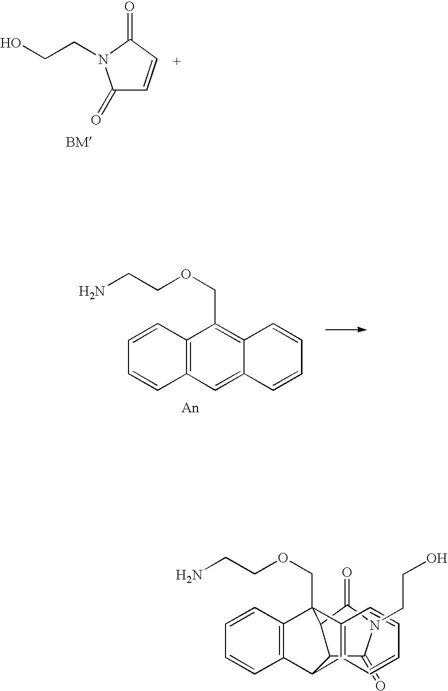

Further, in the case where the target reaction is the amide condensation reaction, examples of the raw substance of reaction include compounds having an amino group in a portion thereof, compounds having a hydroxy group in a portion thereof, and the like.

In the amide condensation, amide is formed by dehydration condensation between the amino group in the compound having an amino group in a portion thereof and the hydroxy group in the compound having a hydroxy group in a portion thereof.

As the reactive modified nucleoside capable of generating the amide condensation, compounds having the structure represented by the Formula E, and the like are exemplified.

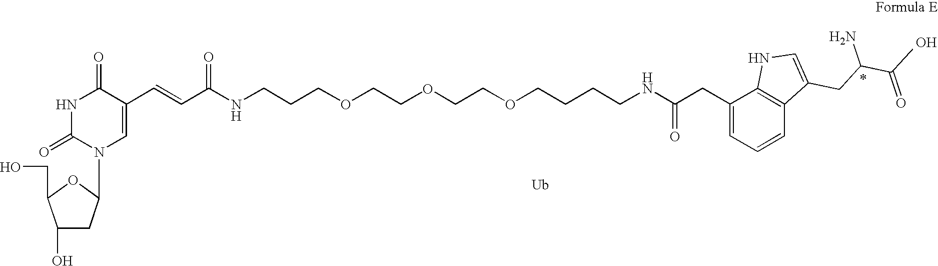

When two above-mentioned reactive modified nucleoside, i.e., two compounds having the structure represented by the Formula E exist, the following amide condensation reaction occurs between the amino group of one compound and the hydroxy group of the other compound, for example, under the presence of ATP and pyrophosphatase.

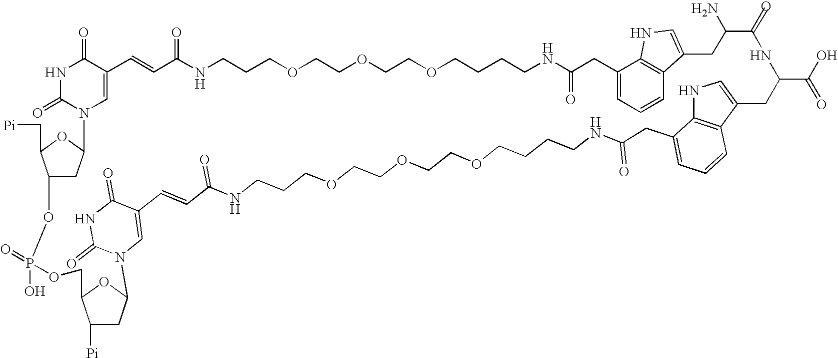

When the reactive modified nucleoside is the compound having the structure represented by the Formula E, for example, molecules having at one end, a group capable of forming an amide condensation and at the other end, a biotin structure which allows separation by adsorption using an avidin column, can be suitably used as the selecting unit.

When the reactive modified nucleoside is the compound having the structure represented by the Formula E, the artificial enzyme, in which the reactive modified nucleoside of the artificial enzyme precursor having the reactive modified nucleoside is substituted with a non-reactive modified nucleoside, can catalyze the following amide condensation reaction. Namely, the target reaction is the amide condensation reaction and the compound shown on the left side of the reaction formula is the raw substance of reaction.

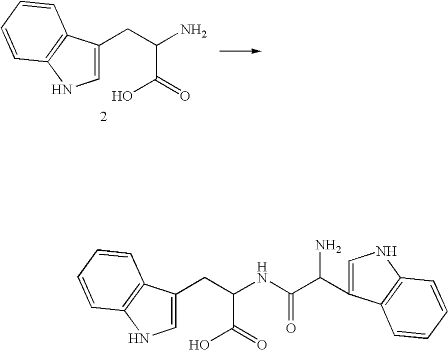

In this amide condensation reaction, two molecules of the compound described on the left side are reacted through condensation reaction to thereby yield the product of the amide condensation reaction described on the right side. As shown in the following Formula F, the product of the amide condensation reaction has asymmetric carbons within a molecule.

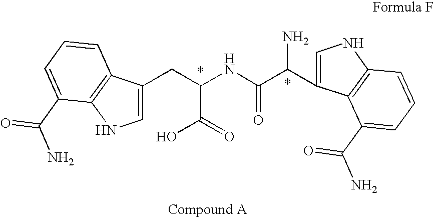

In the Formula F, “*” represents an asymmetric carbon.

Further, as the raw substance of reaction, for example, when the target reaction is an amide bond hydrolysis reaction, compounds having an amide bond in a portion thereof and the like are exemplified.

In the amide bond hydrolysis reaction, the amide group in the compound having the amide group in a portion thereof is hydrolyzed to produce a compound having an amino group in a portion thereof and a compound having a hydroxy group in a portion thereof.

The amide bond hydrolysis reaction may be a mechanism in which reaction starts by the action of trigger, in this case, the trigger is, for example, preferably at least one of ion concentration change, temperature change, and pH change. Examples of the ion concentration change include addition of magnesium ion, and the like. Examples of the temperature change include adjustment to optimal temperature by heating, and the like. Examples of the pH change include adjustment to optimal pH by the addition of a pH adjuster.

As the reactive modified nucleoside capable of generating the amide bond hydrolysis reaction, the compound having the structure represented by the Formula G, or the like are exemplified.

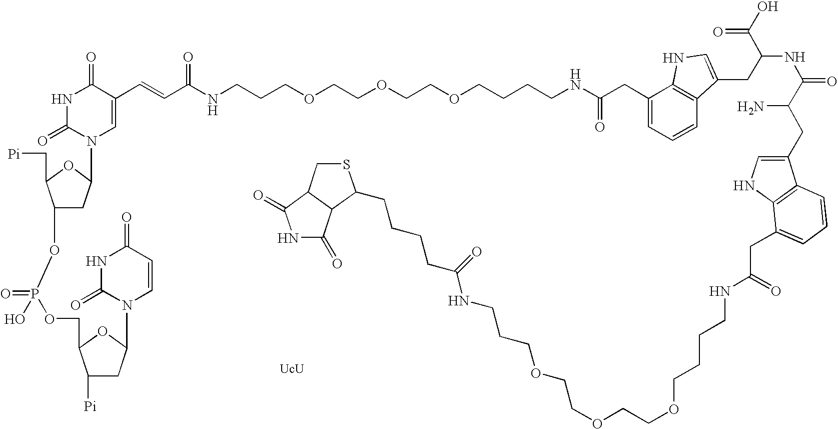

The reactive modified nucleoside, i.e., the compound having the structure represented by the Formula G undergoes the following amide bond hydrolysis reaction under the presence of magnesium ion, etc.

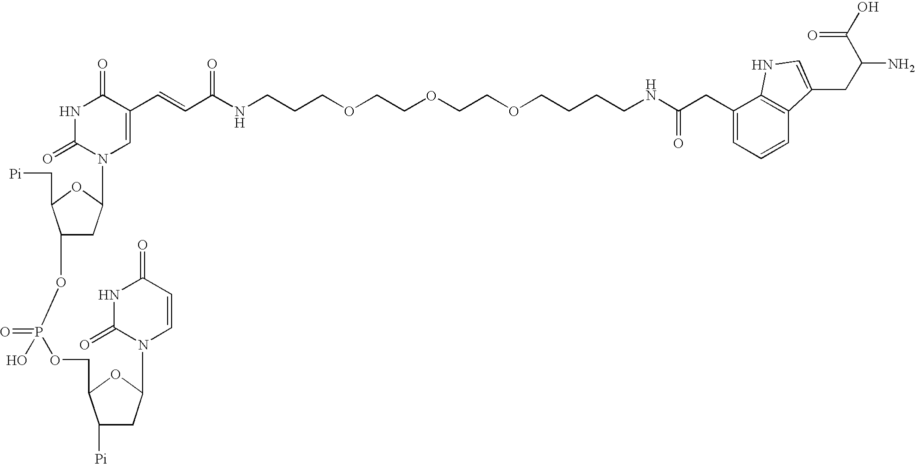

While the reactive modified nucleoside is adsorbed to an avidin column before the amide bond hydrolysis reaction, the reactive modified nucleoside detaches from the avidin column after the amide bond hydrolysis reaction. Thus, the compound having the structure represented by the Formula G, which originally has the biotin structure capable of reacting with the avidin, itself functions as the selecting unit.

When the reactive modified nucleoside is the compound having the structure represented by the Formula G, the artificial enzyme, in which the reactive modified nucleoside of the artificial enzyme precursor having the reactive modified nucleoside is substituted with a non-reactive modified nucleoside, can catalyze the following amide bond hydrolysis reaction. Namely, the target reaction is the amide bond hydrolysis reaction and the compound shown on the left side of the reaction formula is the raw substance of reaction.

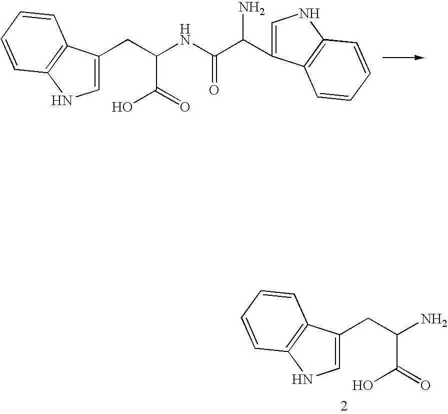

In this amide bond hydrolysis reaction, the compound having an amide bond, which is described in the left side, produces two molecules having an amino group and hydroxy group, which is described in the right side, by the amide bond hydrolysis reaction.

The number, position, etc., of the reactive modified nucleoside in the oligonucleotide sequence is not particularly limited and may be properly selected depending on the application.

The number of the reactive modified nucleosides in the oligonucleotide sequence may be at least one, but may be one or two or more, preferably in the order of one to two. Even if the number is large, it is substantially impossible to sort oligonucleotide sequences reacted at one portion and those reacted at plural portions at the same time.

The position of the reactive modified nucleoside in the oligonucleotide sequence is not particularly limited and may be properly selected depending on the application. For example, the place may be terminus of the oligonucleotide sequence, and may be site other than terminus. However, considering 3 dimensional conformation as the artificial enzyme precursor, the place is preferably site other than terminus and around the central in that the reactivity of the reactive modified nucleoside (enzyme activity of the artificial enzyme) with the raw substance of reaction is excellent, in other words, in that, the place to react with the raw substance of reaction can be formed or provided, and reaction activity (enzyme activity) can be exhibited or improved.

In a conventional design of an artificial enzyme, it was technically difficult to introduce an enzyme's active site into the central part of the chain molecule forming the artificial enzyme, therefore, the enzyme's active site must have been introduced at the terminus of the chain molecule. In the invention; however, since the molecule, which forms the artificial enzyme, is the oligonucleotide sequence, the reactive modified nucleoside may be easily introduced, besides, the oligonucleotide sequence itself can be copied or amplified easily. Thus, the artificial enzyme of the invention is much more advantageous than the conventional artificial enzyme in terms of design, sorting, production, amplification, or the like.

The reactive modified nucleoside may, for example, be labeled with a labeling substance or the like for allowing a convenient selection. The labeling substance is not particularly limited and may be properly selected depending on the application. Examples thereof include radioactive isotopes, chemiluminescence materials, fluorescent materials, enzymes, antibodies, ligands, receptors, and the like. These may be used alone, or two or more may be used in combination.

—Selection of Artificial Enzyme Precursor—

The artificial enzyme precursor can be selected by any method without limitation, and the method may be properly selected depending on the application.

It is preferable that the raw substance of reaction has the capture site which is capable of being captured by the capturing unit provided on the selecting unit. The capture site is not particularly limited as long as it can be captured by the capturing unit, and may be properly selected according to the types, etc. of the capturing unit. Examples of the capture site include antigens, antibodies, enzymes, enzyme substrates, hosts of clathrate compounds, guests of clathrate compounds, and the like. The number, position, etc. of the capture site in the raw substance of reaction is not particularly limited and may be properly selected depending on the application. For example, the number of the capture sites may be one, or may be two or more, and the position of the capture site may be an end of molecule of the raw substance of reaction or may be a portion other than the end.

The capturing unit provided on the selecting unit is not particularly limited as long as it can capture the capture site. Examples thereof include antigens, antibodies, enzymes, enzyme substrates, hosts of clathrate compounds, guests of clathrate compounds, and the like.

The selecting unit is not particularly limited and may be properly selected depending on the application. Examples thereof include columns, beads, separation membranes, network structure, and the like. The number of the capturing units to be introduced into the selecting unit, position at which the capturing unit is introduced, etc. are not particularly limited and may be properly selected depending on the application. For example, the number of the capturing units to be introduced may be one or may be two or more per one selecting unit. Further, the position at which the capturing unit is introduced may be the entire surface of the selecting unit, or may be a part of the surface.

The combination of capturing unit provided on the selecting unit and the capture site provided on the raw substance of reaction is not particularly limited and may be properly selected depending on the application. Examples of the combination include of a combination in which one is avidin, and the other is biotin, and the like.

As a specific method for selecting the artificial enzyme precursor, for example, a method which is performed using the capturing unit provided on the selecting unit such as a column and bead is preferable. Specifically, for example, suitable methods are as follows. The capture site of the raw substance of reaction having the capture site is first captured on the selecting unit, a sample containing the artificial enzyme precursor is then subjected to the selecting unit, and only the artificial enzyme precursor exhibiting reactivity with the raw substance of reaction, i.e., the artificial enzyme precursor comprising the reactive modified nucleoside and exhibiting reactivity with the raw substance of reaction is captured to the raw substance of reaction, thereby selecting a desired artificial enzyme precursor from the sample. Alternatively, the raw substance of reaction and artificial enzyme precursor is allowed to react and then the capture site of the raw substance of reaction reacted with the artificial enzyme precursor is captured by the capturing unit provided on the selecting unit, thereby selecting a desired artificial enzyme precursor from the sample containing the artificial enzyme precursor.

The selection of the artificial enzyme precursor includes a case in which the artificial enzyme precursor is selected from a sample containing one kind of the artificial enzyme precursor, and besides, a case in which desired or all of the artificial enzyme precursors are selected from a sample containing two or more kinds of the artificial enzyme precursors.

In the latter case, as the sample containing two or more kinds of the artificial enzyme precursors, for example, the above-mentioned random polymer pool (random artificial enzyme precursor pool) which comprises two or more kinds of the artificial enzyme precursors, each containing a different oligonucleotide sequence is preferable.

When the artificial enzyme precursor is selected from the random polymer pool (random artificial enzyme precursor pool), usually, two or more artificial enzyme precursors are selected. Usually, when artificial enzymes are produced based on the two or more artificial enzyme precursors selected here, the artificial enzymes exhibit different enzyme activities each other. Thus, it is advantageous to use the random polymer pool (random artificial enzyme precursor pool) in that artificial enzymes exhibiting a desired enzyme activity can be obtained.

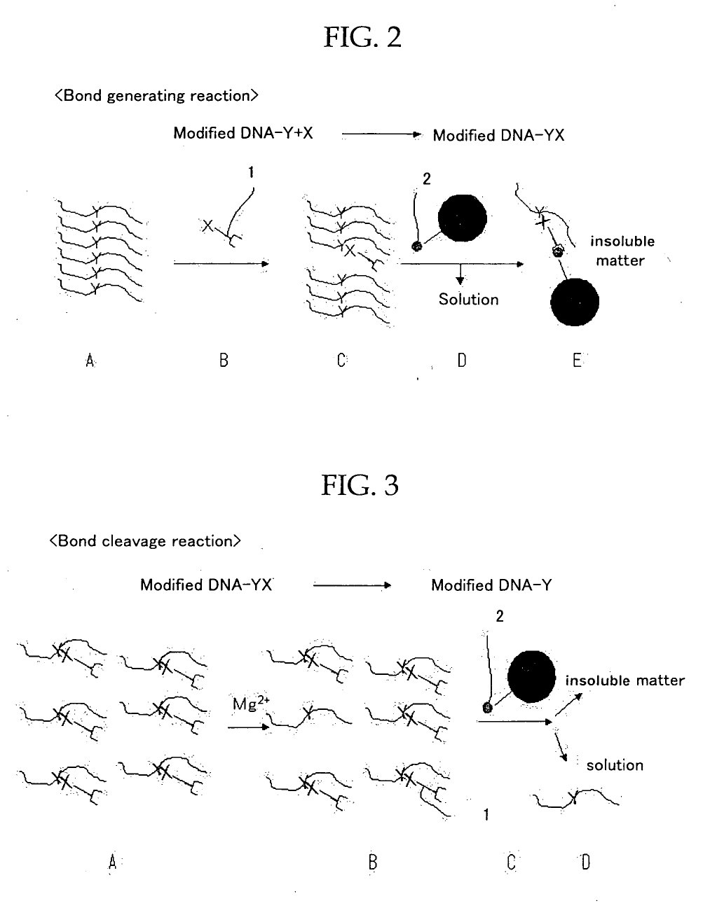

Here, with reference to FIGS. 2A to 2E, a specific example of selection of the artificial enzyme precursor will be explained. The random polymer pool (random artificial enzyme precursor pool) is prepared in advance which contains oligonucleotide sequences, each containing a reactive modified nucleoside Y capable of generating, in case where the target reaction is a bonding reaction, the bonding reaction (e.g., the Diels-Alder reaction, the amide condensation reaction) (FIG. 2A). The random polymer pool is then allowed to react with a solution containing the raw substance of reaction which has a site X capable of generating the bonding reaction 1 with the reactive modified nucleoside Y and which contains a biotin structure as the capture site (FIG. 2B). The reactive modified nucleoside Y and the site X undergo bonding reaction to form a chemical bond XY. As a result, a reaction solution containing the oligonucleotide sequences having the chemical bond XY (FIG. 2C). This reaction solution is then passed though an affinity column in which a resin bead is filled using the resin bead to which avidin 2 capable of capturing the biotin structure as the capture site is fixed, leaving to stand under given conditions (FIG. 2D).

Unlike in the case of oligonucleotide sequences which does not contain the reactive modified nucleoside Y, or oligonucleotide sequences which contain the reactive modified nucleoside Y but exhibit remarkably low reactivity with the raw substance of reaction, in the case of the oligonucleotide sequences which contain the reactive modified nucleoside Y and exhibits reactivity with the raw substance of reaction, as a result of generation of the chemical bond XY, the biotin structure of the raw substance of reaction as the capture site is captured (bound) to avidin as the capturing unit which is fixed to a resin bead as the selecting unit, the oligonucleotide sequences containing the reactive modified nucleoside Y is adsorbed to the affinity column to be insolubilized (FIG. 2E). The affinity column is washed fully with a buffer solution, and thereby oligonucleotide sequences containing no reactive modified nucleoside Y, or oligonucleotide sequences containing the reactive modified nucleoside Y but exhibiting remarkably low reactivity with the raw substance of reaction can be removed. Next, a diluted buffer solution is run through the column, and thereby oligonucleotide sequences containing the reactive modified nucleoside Y and exhibiting reactivity with the raw substance of reaction can selectively selected (collected).

In contrast, when the target reaction is not the bonding reaction, but the breakdown reaction (e.g., the amide bond hydrolysis reaction), the selection can be performed as in the selection of the oligonucleotide sequences when the target reaction is the bonding reaction, shown in FIGS. 3A to 3D. The oligonucleotide sequences containing the reactive modified nucleoside Y and exhibiting reactivity (amide condensation) with the raw substance of reaction can selectively selected (collected) (FIG. 3A), and then, the random polymer pool (random artificial enzyme precursor pool) is subjected to the trigger to cause amide bond hydrolysis reaction (FIG. 3B; the reference character “2” represents biotin). This reaction solution is then is run through an affinity column which is filled with resin beads (column matrix) as the selecting unit, to which avidin as the capturing unit is fixed, leaving to stand under given conditions (FIG. 3C). As a result, the oligonucleotide sequences which has undergone the amide bond hydrolysis reaction are eluted from the affinity column, but the oligonucleotide sequences which has undergone the amide bond hydrolysis reaction remains to be adsorbed to the affinity column, i.e., remains insoluble, and removed (FIG. 3D). And the eluate from the affinity column is collected and from this eluate, the oligonucleotide sequence (the artificial enzyme precursor) exhibiting reactivity with the amide bond hydrolysis reaction can be selected. As mentioned above, examples of the trigger include ion concentration change, temperature change, and pH change.

—Selecting Step—

The sorting step can be performed according to necessity after the artificial enzyme precursor selecting step and before the artificial enzyme producing step which will be mentioned later, and is a step in which from the artificial enzyme precursors selected by the artificial enzyme precursor selecting step, those having high reactivity with a raw substance of reaction is sorted.

The sorting method is not particularly limited and may be properly selected from known methods depending on the application. Examples thereof include various methods such as an affinity chromatography, filter binding, liquid-liquid partition, filtration, gel shift, density gradient centrifugation. These methods may be conducted alone and two or more may be conducted in combination. Among these methods, the affinity chromatography is preferable. In the selection, it is preferable that the dissociation constant between the modified nucleoside of the artificial enzyme precursor (the oligonucleotide sequence) and raw substance of reaction exhibiting reactivity with the modified nucleoside is monitored. In this case, the artificial enzyme precursor having a desired dissociation constant can be selected at one processing at minimum. The artificial enzyme precursor exhibiting a desired reactivity (enzyme activity) can be efficiently selected by measuring the dissociation constant and controlling the dissociation constant. The dissociation constant can be properly selected according to the types, etc. of the raw substance of reaction and can be measured, for example, by using a measuring instrument using surface plasmon resonance.

—Oligonucleotide Sequencing Step—

The oligonucleotide sequencing step can be performed according to necessity after the artificial enzyme precursor selecting step before the artificial enzyme producing step which will be mentioned later, and is a step in which the oligonucleotide sequence of the artificial enzyme precursor selected in the artificial enzyme precursor selecting step.

By the oligonucleotide sequencing step, the oligonucleotide sequence which forms the artificial enzyme precursor is sequenced, making it possible to copy or amplify the oligonucleotide sequence.

In the sequencing step, according to necessity, the oligonucleotide sequence, selected by the artificial enzyme precursor selecting step or sorted by the sorting step, is amplified.

The amplification method is not particularly limited as long as it can amplify the oligonucleotide sequence, and may be selected properly from methods known in the technical field. Examples of the amplification method include a PCR (Polymerase Chain Reaction) method, LCR (Ligase Chain Reaction) method, 3SR (Self-sustained Sequence Replication) method, SDA (Strand Displacement Amplification) method, RT-PCR method, ICAN method, LAMP method, and the like. These methods may be conducted alone or two or more may be conducted in combination.

The PCR method means Polymerase Chain Reaction method and the method can amplify the specific oligonucleotide region by some hundred thousand folds by repeating the DNA synthesis reaction in vitro using DNA synthetase. According to the PCR method, the primer elongation reaction is carried out by allowing the primer to take in 4 kinds or 5 kinds of nucleotide triphosphate (deoxyadenosine triphosphate, deoxyguanosine triphosphate, deoxycytidine triphosphate, and deoxythymidine triphosphate, thymidine triphosphate or deoxyuridine triphosphate (a mixture of these compounds may be also called dNTP) as a substrate.

For carrying out this elongation reaction, an amplification reaction reagent containing the above-mentioned nucleic acid unit and a nucleic acid elongation enzyme is usually used for amplifying a nucleic acid chain. In this case, the usable nucleic acid elongation enzymes include arbitrary DNA polymerases such as E. coli DNA polymerase I, Klenow fragment of E. coli DNA polymerase I, T4 DNA polymerase and the like. Particularly, heat-stable DNA polymerases such as Taq DNA polymerase, Tth DNA polymerase, Vent DNA polymerase and the like can be preferably used. By using these enzymes, there is no necessity of adding a new enzyme for every cycle to enable to repeat the cycle automatically and further to set an annealing temperature to 50° C. to 60° C. Thus, the specificity of target base sequence recognition by the primer can be increased and the gene amplification reaction can be quickly and specifically carried out (Japanese Patent Application Laid-Open UP-A) Nos. 01-314965 and 01-252300).

At carrying out this reaction, an oil can be added to prevent evaporation of water contained in a reaction solution. In this case, the oil may be one such that can be separated from water and has a smaller specific gravity than that of water. Specifically, silicone oil, mineral oil and the like are exemplified. Further, according to a gene amplification instrument, such the medium is not needed, and the primer elongation reaction can be also conducted by using such the gene amplification instrument.

As described above, repeating the elongation reaction by using the primer allows efficient gene amplification of the oligonucleotide of interest to yield a large amount of the oligonucleotide sequence. The specific method including conditions for the gene amplification reaction includes the known methods described in references such as “Jikken Igaku” (Yodosha, 8, No. 9. 1990), “PCR Technology” (Stockton Press, 1989), and the like.

By carrying out the PCR method, the oligonucleotide sequence is substituted with a natural oligonucleotide sequence which is not modified with a substituent.

When the oligonucleotide sequence is not the DNA but the RNA, the DNA can be synthesized by performing a reverse transcription reaction. The reverse transcription reaction is a process for synthesizing the DNA by using the RNA as a template. A reaction solution and a reaction condition of the reverse transcription reaction are different in accordance with a target RNA. For example, operations comprise adding an RNase-free sterilized distilled water and a 3′-primer to an RNA solution and incubating and, then, cooling and adding a reverse transcription buffer solution, which includes Tris-HCl, KCl, MgCl₂ and the like, DTT, and dNTPs, and adding a reverse transcriptase followed by incubation. Stopping the reverse transcription reaction can be operated by adjusting conditions of the incubation. Such the reverse transcription reaction can be also performed by a reverse transcription PCR.

In the sequencing step, without amplifying or, if required, after amplification of the oligonucleotide sequence, the base sequence of the oligonucleotide sequence is determined.

The process for determining the base sequence is not particularly limited and can be selected properly from methods known in the art depending on the application. Examples of the method include a method by gene cloning, chain terminator method, Sanger method, a DNA sequencer (automated DNA base sequence determination equipment) by using a dideoxy method, and the like. These methods may be employed alone or two or more may be employed.

—Translating Step—

The translating step is a step in which the oligonucleotide sequence (base sequence), which has been sequenced or determined in the oligonucleotide sequencing step, is translated on the basis of the relation table prepared by relating at least one kind of 4ⁿ nucleotide n-mers, which are presented in the relation table prepared by the one-to-one combination of 4 nucleosides, to one kind of nucleotide n-mers.

The translation is preferably carried out for every n base(s) from the 5′ terminal side of the oligonucleotide sequence made of the nucleotide n-mer, of which base sequence has been determined, on the basis of the relation table. For example, when the modified nucleotide n-mer is a nucleotide dimer, the translation is preferably carried out for every 2 bases from the 5′ terminal side of the oligonucleotide sequence, of which base sequence has been determined, made of the nucleotide dimer on the basis of the relation table (e.g., following relation table).

TABLE 3

5′

| 3′ | A | C | G | T |
|---|---|---|---|---|
| A | — | C₂A | — | U₃A |
| C | AC₁ | C₃C | GC₆ | U₄C |
| G | — | C₄G | — | U₅G |
| T | AU₁ | C₅T | GU₂ | U₆T |

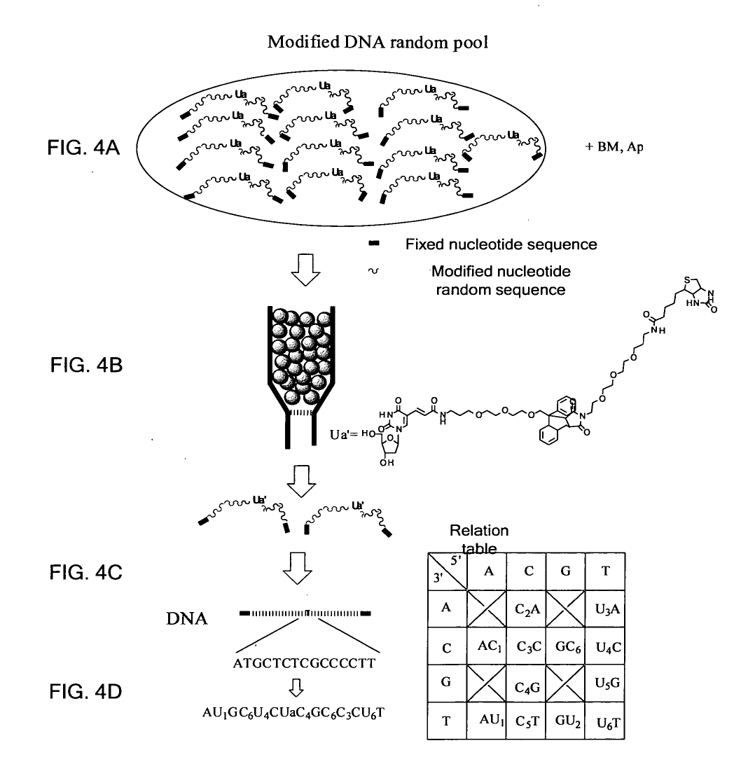

For example, as shown in FIG. 4D, when the determined modified oligonucleotide sequence is “ATGCTCTAGCCCCT,” it is confirmed that the sequence is translated into “AU₁GC₆U₄CU₃AGC₆C₃CC₅T” on the basis of the relation table. In this way, the base sequence of the artificial enzyme precursor (oligonucleotide sequence) can be determined.

When the nucleotide n-mer is a nucleotide trimer, the translation is carried out for every 3 bases from the 5′ terminal side of the oligonucleotide sequence composed of the nucleotide trimer, of which base sequence has been determined, on the basis of the relation table (e.g., the following relation table).

TABLE 4

| | A | | C | | G | | T | |
|---|---|---|---|---|---|---|---|---|
| A | A | G | A | G | A | G | A | G |
| | — | — | C₂AA | C₃AG | — | — | U₃AA | U₄AG |
| | C | T | C | T | C | T | C | T |
| | AAC₁ | AAU₁ | C₄AC | C₅AT | GAC₆ | GAU₂ | U₅AC | U₆AT |
| C | A | G | A | G | A | G | A | G |
| | AC₇A | AC₈G | C₁₁CA | C₁₂CG | GC₁₅A | GC₁₆G | U₇CA | U₈CG |
| | C | T | C | T | C | T | C | T |
| | AC₉C | AC₁₀T | C₁₃CC | C₁₄CT | GC₁₇C | GC₁₈T | U₉CC | U₁₀CT |
| G | A | G | A | G | A | G | A | G |
| | — | — | C₂₀GA | C₂₁GG | — | — | U₁₃GA | U₁₄GG |
| | C | T | C | T | C | T | C | T |
| | AGC₁₉ | AGU₁₁ | C₂₂GA | C₂₃GT | GGC₂₄ | GGU₁₂ | U₁₅GC | U₁₆GT |
| T | A | G | A | G | A | G | A | G |
| | AU₁₇A | AU₁₈G | C₂₅TA | C₂₆TG | GU₂₁A | GU₂₂T | U₂₅TA | U₂₆TG |
| | C | T | C | T | C | T | C | T |
| | AU₁₉C | AU₂₀T | C₂₇TC | C₂₈TT | GU₂₃C | GU₂₄T | U₂₇TC | U₂₈TT |

When the nucleotide n-mer is a nucleotide tetramer or higher nucleotide (i.e., n≧4), the translation is also carried out in the same way, for example, on the basis of the relation table prepared by relating at least 1 kind of 4ⁿ kinds of nucleotide n-mers (n≧4), which are represented in the relation table prepared by the one-to-one combination of 4 kinds of nucleosides, to 1 kind of the nucleotide n-mer (n≧4).

—Artificial Enzyme Producing Step—

The artificial enzyme producing step is a step in which an artificial enzyme is produced which can catalyze the target reaction and comprises an oligonucleotide sequence (may be referred to as “normal oligonucleotide sequence”) in which the reactive modified nucleoside of the artificial enzyme precursor is substituted with a non-reactive modified nucleoside which is non-reactive with the raw substance of reaction.

The non-reactive modified nucleoside is, for example, a nucleoside in which the substituent or a portion containing the substituent is removed from the reactive modified nucleoside present in the artificial enzyme precursor (the oligonucleotide sequence). For example, when the reactive modified nucleoside is a uridine derivative, uridine is preferable.

The substitution with the non-reactive modified nucleoside, in other words, production or formation of the artificial enzyme (the normal oligonucleotide sequence) comprising the non-reactive modified nucleoside is carried out, in the same manner as in the case of the production or formation of the artificial enzyme precursor (the oligonucleotide sequence comprising the reactive modified nucleoside), by using the non-reactive modified nucleoside corresponding to the reactive modified nucleoside instead of the reactive modified nucleoside which is the monomer at the time of forming an oligonucleotide sequence.

The artificial enzyme obtained by the artificial enzyme producing step is the artificial enzyme according to the invention and has a desired enzyme activity to the target reaction.

The artificial enzyme according to the invention obtained by the method for producing an artificial enzyme of the invention has high reaction specificity and provides improved reaction efficiency, is stable, and can be suitably used in a variety of applications as an artificial enzyme based on nucleic acids.

The artificial enzyme according to the invention may comprise, for example, a functional molecule functioning as an antibody in a portion thereof.

The functional molecule is not particularly limited and can be properly selected depending on the application. For example, a functional molecule is suitable which comprises a linker containing a first nucleic acid sequence at one end and a second nucleic acid sequence at the other end; a first target capturing part containing a first complementary nucleic acid sequence capable of complementarily binding to the first nucleic acid sequence; and a second target capturing part containing a second complementary nucleic acid sequence capable of complementarily binding to the second nucleic acid sequence.

In case of the functional molecule, the first and second target capturing parts can preferably capture different points within one target. Further, the first and second target capturing parts of the functional molecule is preferably a nucleotide polymer containing a modified nucleoside in which a substituent is introduced into a nucleoside constituting nucleic acids. Further, in the functional molecule, the site of the linker excluding the first and second nucleic acid sequences is preferably formed of an arbitrary nucleic acid.

The type of the artificial enzyme according to the invention is not particularly limited. Examples thereof include oxidoreductases, transferases, hydrolases, lyases, isomerases, synthetases, and the like. The artificial enzyme according to the invention not only includes those having enzyme activity but also those having a function promoting chemical reaction, which is achieved in a way that the artificial enzyme itself binds to the transition state (activated state) to be stabilized and thereby the free energy of activation of a reaction is decreased.

The invention will be illustrated in further detail with reference to several examples below, which are not intended to limit the scope of the invention.

### EXAMPLES

### Example 1

In the way shown in FIGS. 4A to 4D, production (synthesis and identification) of the artificial enzyme according to the invention which has enzyme activity was performed. Specifically, first, by the process expressed by the following formula, 6 kinds of deoxycytidine relatives (C₁₋₆) having a functional group at the 5th position of cytosine and 6 kinds of deoxyuridine relatives (U₁₋₆) having a functional group at the 5th position of uracil were each synthesized (prepared).

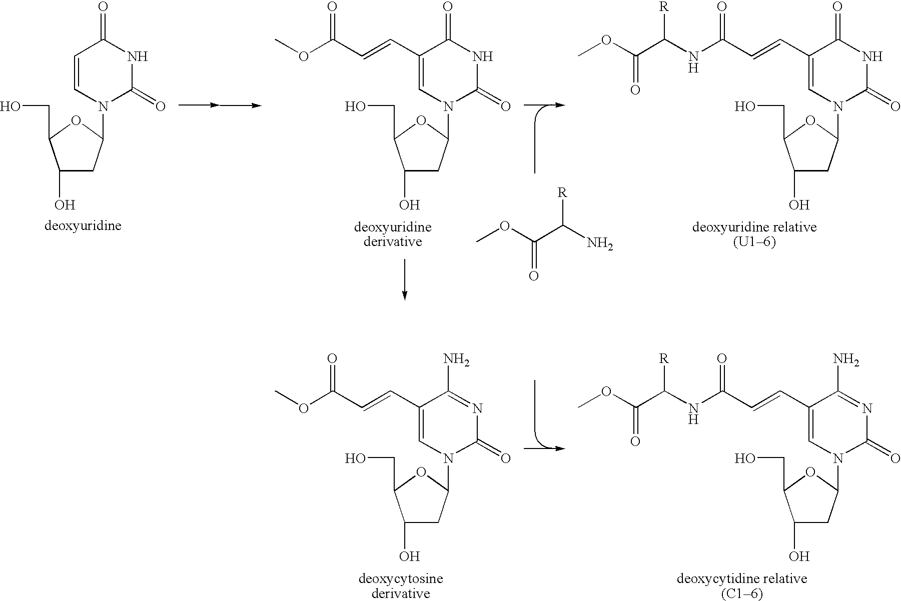

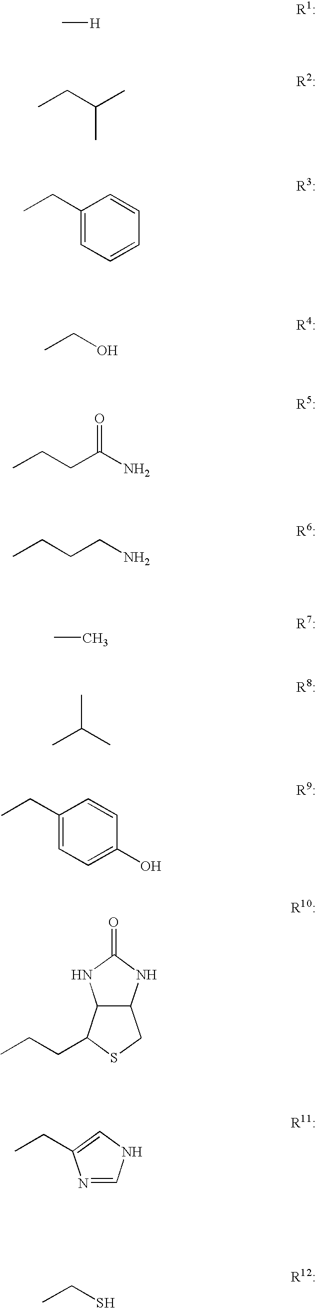

Next, each of the synthesized (prepared) 12 kinds of the modified nucleoside dimer was one-to-one related to any one selected from 16 patterns of the relation table shown below, which is prepared by the one-to-one combination of 4 kinds of nucleosides constituting DNA.

TABLE 5

5′

| 3′ | A | C | G | T |
|---|---|---|---|---|
| A | — | C₂A | — | U₃A |
| C | AC₁ | C₃C | GC₆ | U₄C |
| G | — | C₄G | — | U₅G |
| T | AU₁ | C₅T | GU₂ | U₆T |

Next, 12 kinds of the modified oligonucleotide amidite (M) represented in the above relation table were chemically synthesized by the phosphoramidite method. Specifically, modified nucleotide dimer (AU₁) can be, as shown in the following formula, synthesized from deoxyadenosine and modified deoxyuridine. 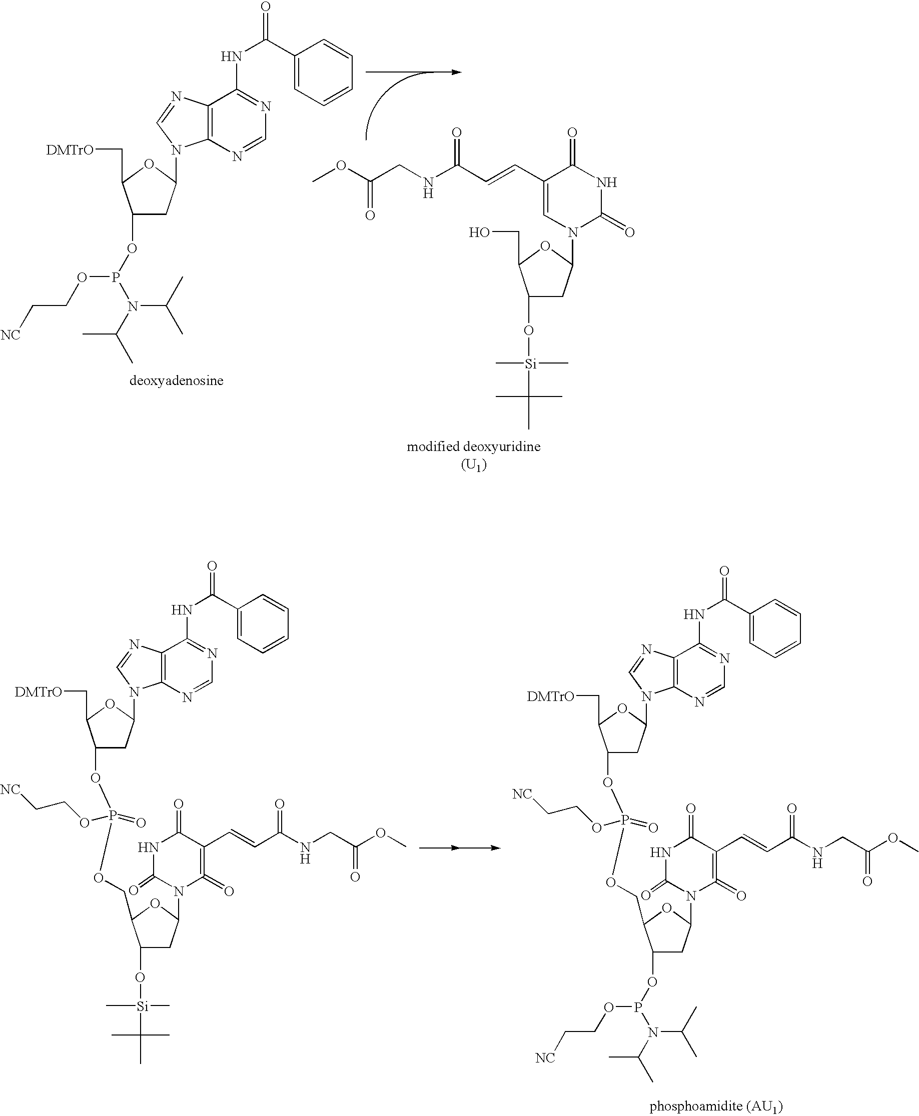
In the formula, DMTr represents a dimethoxytrityl group.

And, by using a DNA synthesizer (available from Applied Biosystems), a random polymer pool (random artificial enzyme precursor pool) was prepared containing oligonucleotide sequence (N₂₀-M₁₀-Ua-M₁₀-N₂₀ (DNA 81-mer)) composed of a fixed oligonucleotide sequence 20-mer (N₂₀)−a modified oligonucleotide random sequence 10-mer (M₁₀)+an anthracene-containing uridine relative represented by the following formula (Ua)+the modified oligonucleotide random sequence 10-mer (M₁₀)−the fixed oligonucleotide sequence 20-mer (N₂₀).

In the oligonucleotide sequence, the anthracene-containing uridine relative (Ua) corresponds to the reactive modified nucleoside. In the anthracene-containing uridine relative (Ua), the uridine portion and the portion other than the uridine corresponds to a nucleoside and the substituent, respectively. The anthracene, which is present at the end of the substituent, is one of the raw substance of reactions of Diels-Alder reaction which is the target reaction.

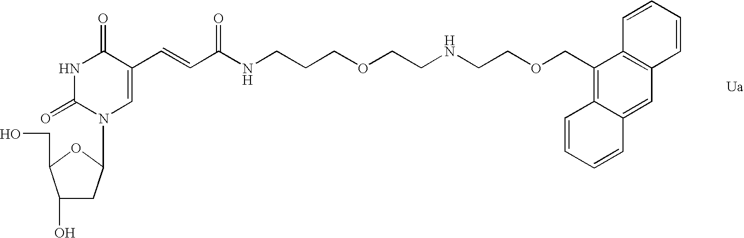

Next, a mixed solution of obtained random polymer pool (random artificial enzyme precursor pool) (FIG. 4A), 1 mM biotinylated maleinimide (BM) represented by the following formula, and 1 mM anthracene derivative (Ap) represented by the following formula was left to stand at room temperature (25° C.) overnight to be allowed to react. The content of the oligonucleotide sequence in the random polymer pool was 0.001 mM. The biotin structure in the biotinylated maleinimide (BM) corresponds to the capture site, and the biotin structure as the capture site can be captured by avidin as the capturing unit. The avidin as the capturing unit was fixed on the surface of a resin bead as the selecting unit.

The anthracene portion of the anthracene-containing uridine relative (Ua), which is the reactive modified nucleoside of the oligonucleotide sequence, and imide ring portion of the biotinylated maleinimide (BM) underwent the Diels-Alder reaction (binding reaction). At this time, the oligonucleotide which does not contain the anthracene-containing uridine relative (Ua), or the oligonucleotide which contains the Ua but has remarkably low reactivity with the anthracene portion did not cause the Diels-Alder reaction.

The reaction solution after such a Diels-Alder reaction was passed through an affinity column filled with resin beads (SOFTLINK Soft Release Avidin Resin, available from Promega Corporation) to which the avidin as the selecting unit is bound and was left to stand under given conditions (for 30 minutes at room temperature). Consequently, among the oligonucleotide sequences, those of which anthracene-containing uridine relative (Ua), the reactive modified nucleoside, reacted with the biotinylated maleinimide (BM) are adsorbed to the affinity column because the biotin structure as the capture site of the biotinylated maleinimide (BM) is captured by the avidin as the capturing unit, which is fixed to the resin bead as the selecting unit (FIG. 4B). Thereafter, the oligonucleotide sequence adsorbed to the affinity column was eluted (5 mM biotin) from the affinity column. In this way, an artificial enzyme precursor (oligonucleotide sequence), capable of generating the Diels-Alder reaction, was selected. This is the “artificial enzyme precursor selecting step”.

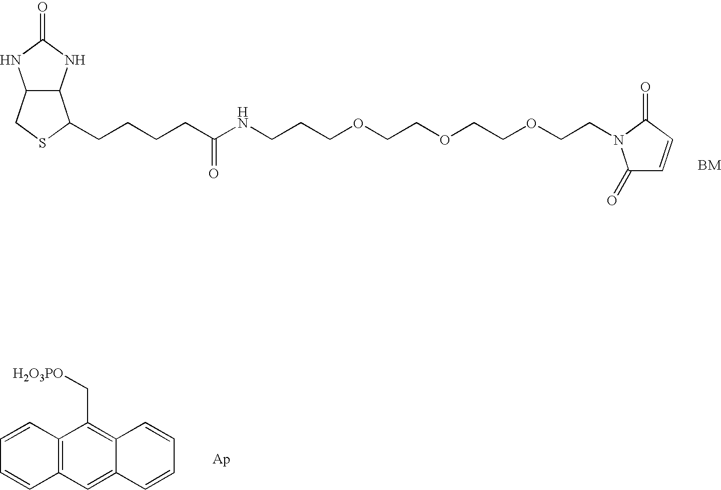

Next, the selected oligonucleotide sequence was amplified by PCR and the base sequence thereof was determined by cloning, resulting that a part of the base sequence of the oligonucleotide was “ATGCTCTAGCCCCT” (FIG. 4C). This is the “oligonucleotide sequencing step”.

This base sequence was translated based on the above-mentioned relation table to obtain “AU₁GC₆U₄CU₃AGC₆C₃CC₅T”. In this way, the structure of the selected artificial enzyme precursor (the oligonucleotide sequence) was identified (FIG. 4D). This is the “translating step”.

Similarly, with respect to the selected oligonucleotide sequence, 100 clones each were sequenced to determine the base sequence. As a result, it was found that the oligonucleotide sequences obtained from 10 clones of the 100 clones could generate efficiently a reaction product represented by the following formula, resulting from Diels-Alder reaction under the presence of the biotinylated maleinimide (BM).

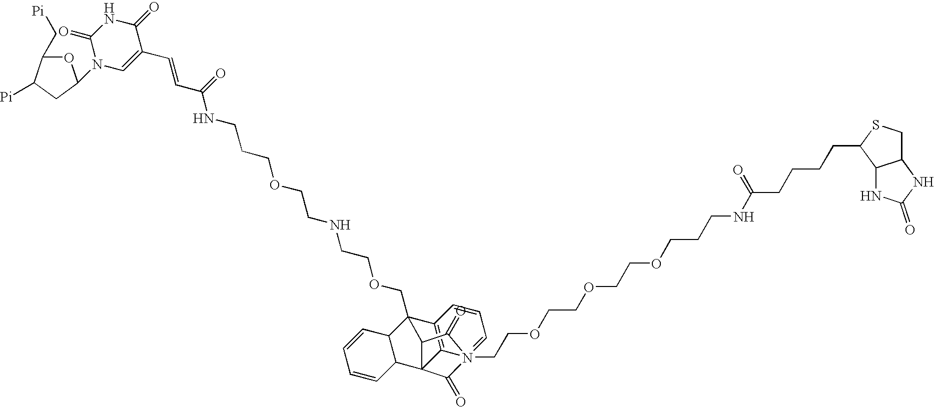

Further, 10 kinds of oligonucleotide sequences (normal oligonucleotide sequences) were synthesized in which Ua of the oligonucleotide sequence (artificial enzyme precursor) obtained from the above-mentioned 10 clones was substituted with uridine (U).

Next, it was found that 2 kinds of these 10 kinds of the oligonucleotide sequences (normal oligonucleotide sequences) accelerated or catalyzed the Diels-Alder reaction represented by the following formula by 1,000 fold to 10,000 fold under the presence of 1 mM 2-hydroxyethyl maleimide (BM′) and 1 mM anthracene derivative (An) (room temperature: 25° C.) which are represented in the following formula. These 2 kinds can be used as an artificial enzyme and are the artificial enzyme according to the invention. This corresponds to the “artificial enzyme producing step”.

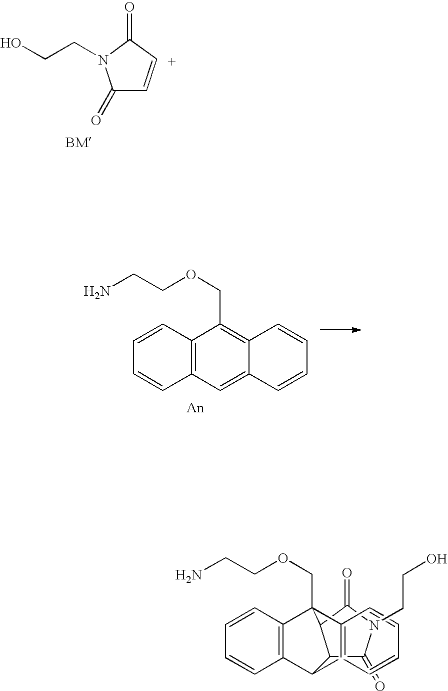

### Example 2

An artificial antibody A which specifically binds to the following compound A was synthesized in accordance with the artificial antibody synthetic method shown in the “functional molecule and process for producing the same” described in International Publication WO03/078623 by this applicant.

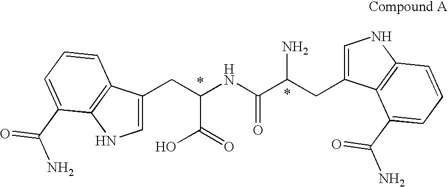

In the formula, “*” represents an asymmetric carbon.

An artificial enzyme catalyzing an amide condensation reaction was produced (synthesized and identified) as follows. First, in the same way as in Example 1, a random polymer pool (random artificial enzyme precursor pool) was prepared containing oligonucleotide sequence (N₂₀-M₁₀-Ub+Ub-M₁₀ -N₂₀ (DNA 82-mer)) composed of a fixed oligonucleotide sequence 20-mer (N₂₀)−a modified oligonucleotide random sequence 10-mer (M₁₀)−an uridine relative (Ub) represented by the following formula+the modified oligonucleotide random sequence 10-mer (M₁₀)−the fixed oligonucleotide sequence 20-mer (N₂₀). This is the “oligonucleotide sequence producing step”.

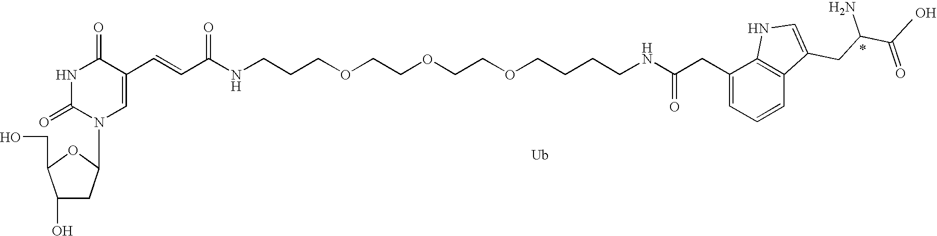

Next, a mixed solution of obtained random polymer pool (content of the oligonucleotide sequence: 0.01 mM), ATP (1 mM), and pyrophosphatase (3 unit) was left to stand at room temperature (25° C.) overnight to be allowed to react.

The structure (see the figure below) which was formed by dehydration condensation between two molecules of the uridine relative (Ub) represented by the formula comprises the above-mentioned compound A structure as the capture site and thus can be captured by an artificial antibody A as the capturing unit. The artificial antibody A as the capturing unit was fixed to the surface of a resin bead as the selecting unit.

The carboxylic acid (—COOH) of the uridine relative (Ub) represented by the formula, which is the reactive modified nucleoside of the oligonucleotide sequence, and the amino group (—NH₂) of another Ub underwent the amide condensation reaction (binding reaction). At this time, the oligonucleotide which does not contain the uridine relative (Ub) represented by the formula, or the oligonucleotide which contains the Ub but has remarkably low reactivity (reaction activity) of the amide condensation reaction did not cause the amide condensation reaction.

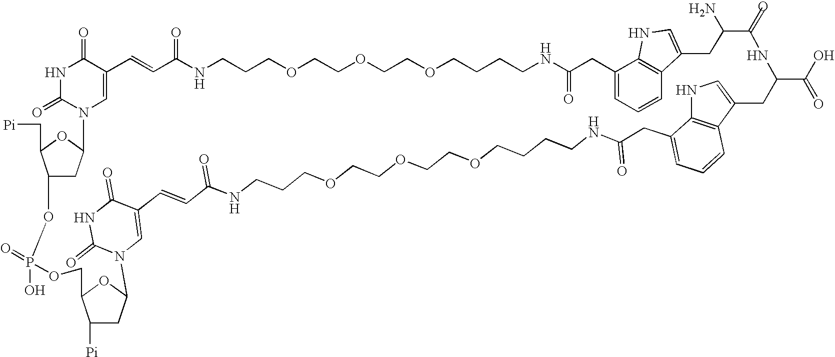

The reaction solution after such an amide condensation reaction was passed through an affinity column filled with resin beads as the selecting unit to which the artificial antibody A is bound and was left to stand under given conditions. Consequently, among the oligonucleotide sequences, with respect to those of which uridine relative (Ub) represented by the formula, the reactive modified nucleoside, underwent the amide condensation reaction, the above-mentioned compound A structure as the capture site was generated. Thus, those which underwent the amide condensation reaction were captured by the artificial antibody A as the capturing unit which was fixed to the resin beads as the selecting unit, and adsorbed to the affinity column. Thereafter, the adsorbed oligonucleotide sequence was eluted (above-mentioned compound A: 5 mM) from the affinity column. In this way, an artificial enzyme precursor (oligonucleotide sequence), which can generate the amide condensation reaction, was selected. This is the “artificial enzyme precursor selecting step”.

Next, the selected oligonucleotide sequence was amplified by PCR and the base sequence thereof was determined by cloning. This is the “oligonucleotide sequencing step”. This base sequence was translated based on the above-mentioned relation table to identify the structure of the selected artificial enzyme precursor (the oligonucleotide sequence). This is the “translating step”. Similarly, with respect to the selected oligonucleotide sequence, 100 clones each were sequenced to determine the base sequence. As a result, it was found that the oligonucleotide sequences obtained from 5 clones of the 100 clones could synthesize efficiently a product represented by the following formula, resulting from amide condensation reaction under the presence of ATP and pyrophosphatase.

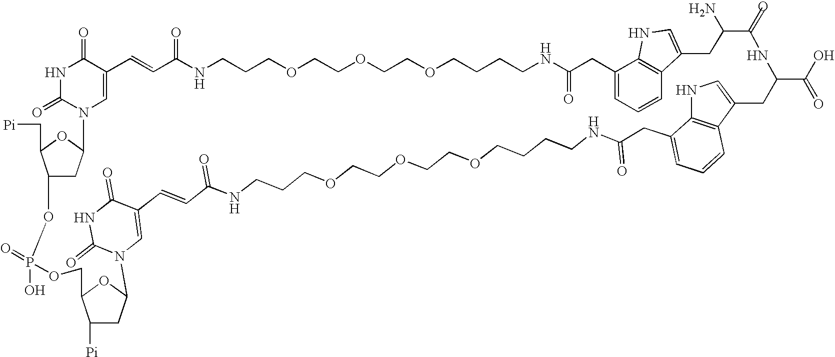

Further, 5 kinds of oligonucleotide sequences (normal oligonucleotide sequence) were synthesized in which Ub of the oligonucleotide sequence (artificial enzyme precursor) obtained from the above-mentioned 5 clones was substituted with uridine (U).

Next, it was found that 2 kinds of these 5 kinds of the oligonucleotide sequences (normal oligonucleotide sequences) accelerated or catalyzed the amide condensation reaction represented by the following formula by 100,000 fold under the presence of tryptophan, ATP, and pyrophosphatase (room temperature: 25° C.). These 2 kinds can be used as an artificial enzyme and are the artificial enzyme according to the invention. This corresponds to the “artificial enzyme producing step”.

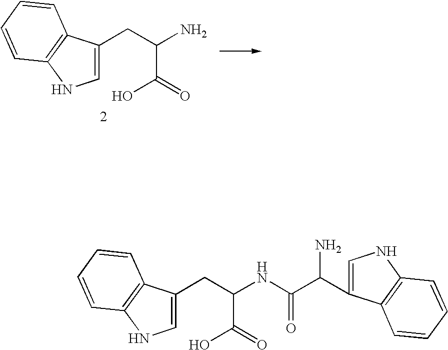

### Example 3

An artificial enzyme precursor (oligonucleotide sequence) was produced (synthesized and identified) in the same way as in Example 2, except that Ub of the artificial enzyme precursor (oligonucleotide sequence) obtained in Example 2 was changed to UcU represented by the following formula.

This oligonucleotide sequence did not exhibit catalytic activity for the amide bond hydrolysis reaction under the coexistence of 10 mM MgCl₂.

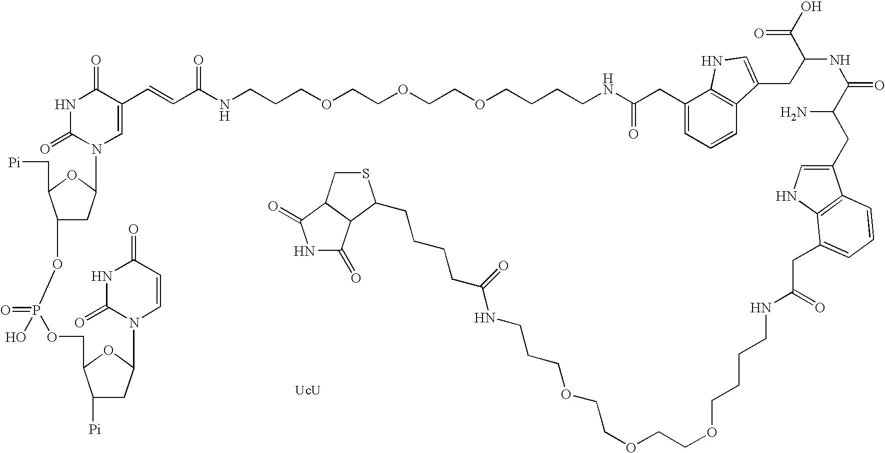

Next, in the same way as in Example 1, a random oligonucleotide N₂₀-M₁₀-Uc+U-M₁₀-N₂₀ (DNA 82-mer) was prepared which is composed of a fixed oligonucleotide sequence 20-mer (N₂₀)−a modified oligonucleotide random sequence 10-mer (M₁₀)−an uridine relative Uc+uridine (U)−the modified oligonucleotide random sequence 10-mer (M₁₀)—the fixed oligonucleotide sequence 20-mer (N₂₀). Subsequently, with respect to the M dimer portion (M₁₀) of the obtained random oligonucleotide, 2% mix block amidite was used as a raw material to thereby prepare a random polymer pool (artificial enzyme precursor).

Next, thus obtained random polymer pool (random artificial enzyme precursor pool) was left to stand overnight under the coexistence of 10 mM MgCl₂ as the trigger to be allowed to react (25° C). This reaction solution was passed through an affinity column filled with resin avidin beads, oligonucleotide sequence containing biotin was adsorbed to the affinity column to be insolubilized. In contrast, eluted solution was passed through an affinity column filled with the resin beads coated with the artificial antibody A, and oligonucleotide sequence containing the above-mentioned compound A structure was insolubilized.

Next, obtained soluble matter was amplified by PCR and the base sequence of the DNA thereof was determined by cloning. Similarly, 100 clones each were sequenced to determine the base sequence. As a result, it was found that the oligonucleotide sequences obtained from 20 clones of the 100 clones could catalyze efficiently the amide bond hydrolysis reaction under the presence of magnesium ion.

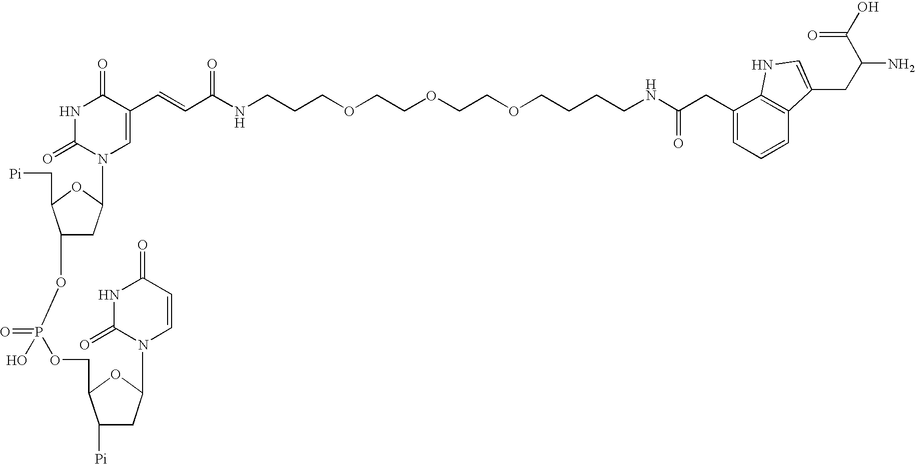

Further, oligonucleotides were synthesized in which the Uc of the oligonucleotide sequence obtained from the above-mentioned 20 clones was substituted with uridine (U).

Next, it was found that the oligonucleotide sequences (normal oligonucleotide sequences) obtained from 3 clones of the 20 clones accelerated the hydrolysis reaction represented by the following formula by 300,000 fold under the presence of tryptophan dimer and magnesium ion. These 3 kinds can be used as an artificial enzyme and are the artificial enzyme according to the invention. This corresponds to the “artificial enzyme producing step”.

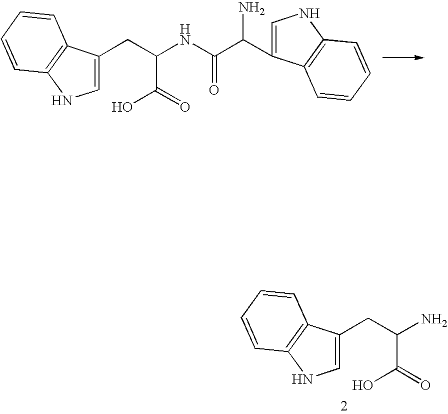

The method for producing an artificial enzyme according to the invention can be suitably used for producing artificial enzymes efficiently and particularly suitably used for producing an artificial enzyme of the invention efficiently.

As mentioned above, the artificial enzyme according to the invention has having a self-replicating ability, is easily copied or amplified, can be mass-produced, is excellent in stability and safety, is easily recovered and has excellent handleability, has excellent versatility, and thus can be suitably used widely in a variety of fields. The artificial enzyme according to the invention can be particularly suitably used in the fields such as pharmaceuticals, drug deliveries, and biosensors. Further, when the artificial enzyme is designed as a multifunctional molecule having both an antibody function and an enzyme function, the artificial enzyme can be particularly suitably used in the fields such as pharmaceuticals, drug deliveries, and biosensors.

The invention can solve the conventional problems and can provide a method for producing an artificial enzyme that can produce easily and efficiently an artificial enzyme which exhibits a desired enzyme activity (catalytic activity) to a desired target reaction without limitation to the type of the target reaction which the artificial enzyme catalyzes and which can be copied; and an artificial enzyme which is produced by the method for producing an artificial enzyme, of which enzyme activity to a desired target reaction can be controlled, which is easily copied or amplified, selectively recovered, and activated, which allows easy screening of enzyme activity to the target reaction, and which is excellent in stability, safety, and handleability.

What is claimed is:

1. A method for producing an artificial enzyme comprising the steps of:
selecting an artificial enzyme precursor which comprises an oligonucleotide sequence containing modified nucleosides prepared by introducing a substituent into each nucleoside and at least one of the modified nucleoside capable of reacting with a raw substance of a target reaction which the artificial enzyme catalyzes; and
producing the artificial enzyme which is capable of catalyzing the target reaction and comprises the oligonucleotide sequence in which the modified nucleoside capable of reacting with the raw substance of the artificial enzyme precursor is substituted with a non-reactive modified nucleoside which is non-reactive with the raw substance of the target reaction.

2. The method for producing an artificial enzyme according to claim 1, wherein, in the step of selecting an artificial enzyme precursor, the artificial enzyme precursor is selected from a random artificial enzyme precursor pool which comprises two or more kinds of the artificial enzyme precursors, each containing a different oligonucleotide sequence.

3. The method for producing an artificial enzyme according to claim 1, further comprising, after the step of selecting the artificial enzyme precursor and before the step of producing the artificial enzyme,
a step of sequencing the oligonucleotide sequence of the artificial enzyme precursor selected in the step of selecting the artificial enzyme precursor.

4. The method for producing an artificial enzyme according to claim 3, further comprising a step of translating the oligonucleotide sequence sequenced in the step of sequencing on the basis of the relation table prepared by relating at least one kind of 4ⁿ nucleotide n-mers, which are presented in the relation table prepared by the one-to-one combination of 4 nucleosides, to one kind of nucleotide n-mers.

5. The method for producing an artificial enzyme according to claim 1, wherein, in the step of selecting the artificial enzyme precursor, two or more kinds of artificial enzyme precursors are selected.

6. The method for producing an artificial enzyme according to claim 5, further comprising, after the step of selecting the artificial enzyme precursor and before the step of producing the artificial enzyme,
a step of producing the artificial enzyme precursor which comprises the oligonucleotide sequence sequenced in the step of sequencing the oligonucleotide sequence; and
a step of sorting the artificial enzyme precursor having high reactivity with the raw substance of reaction from the artificial enzyme precursor produced by the step of producing an artificial enzyme precursor.

7. The method for producing an artificial enzyme according to claim 1, wherein the raw substance of reaction comprises a capture site capable of being captured by a capturing unit, and
wherein the selection in the step of selecting the artificial enzyme precursor is carried out by allowing the raw substance of reaction and the reactive modified nucleoside, and then by capturing the capture site of the raw substance of reaction by the capturing unit to thereby capture the reactive modified nucleoside reacted with the raw substance of reaction.

8. The method for producing an artificial enzyme according to claim 7, wherein the capturing unit is at least one selected from the group consisting of an antigen, an antibody, an enzyme, an enzyme substrate, a host of a clathrate compound, and a guest of a clathrate compound, and
wherein the capture site is at least one selected from the group consisting of the antigen, the antibody, the enzyme, the enzyme substrate, the host of a clathrate compound, and the guest of a clathrate compound, and is capable of being captured by the capturing unit.

9. The method for producing an artificial enzyme according to claim 8, wherein the capturing unit is one of avidin and biotin, and
wherein the capture site is the biotin when the capturing unit is avidin, and the capture site is the avidin when the capturing unit is biotin.

10. The method for producing an artificial enzyme according to claim 7, wherein the capturing unit is fixed to a selecting unit.

11. The method for producing an artificial enzyme according to claim 10, wherein the selecting unit is at least one of a column and a bead.

12. The method for producing an artificial enzyme according to claim 1, wherein the target reaction which the artificial enzyme catalyzes is at least one selected from the group consisting of a bonding reaction and a breakdown reaction.

13. The method for producing an artificial enzyme according to claim 12, wherein the bonding reaction is selected from a polymerization reaction, a condensation reaction, a condensation polymerization reaction, an addition reaction, and a polyaddition reaction, and wherein the breakdown reaction is selected from a hydrolysis reaction.

14. The method for producing an artificial enzyme according to claim 1, wherein the non-reactive modified nucleoside is a uridine and the reactive modified nucleoside is a uridine derivative.

15. The method for producing an artificial enzyme according to claim 1, wherein the non-reactive modified nucleoside is a uridine and the reactive modified nucleoside is a uridine derivative having a structure capable of reacting with the raw substance of reaction in a portion thereof.

16. The method for producing an artificial enzyme according to claim 1, wherein the reactive modified nucleoside in the oligonucleotide sequence of the artificial enzyme precursor is positioned at a portion other than terminus of the oligonucleotide sequence.

17. The method for producing an artificial enzyme according to claim 1, wherein a reaction between the reactive modified nucleoside and the raw substance of reaction is at least one selected from the group consisting of a bonding reaction and a breakdown reaction.

18. The method for producing an artificial enzyme according to claim 17, wherein the bonding reaction is selected from a polymerization reaction, a condensation reaction, a condensation polymerization reaction, an addition reaction, and a polyaddition reaction, and wherein the breakdown reaction is selected from a hydrolysis reaction.

19. The method for producing an artificial enzyme according to claim 1, wherein a reaction between the reactive modified nucleoside and the raw substance of reaction is at least one selected from the group consisting of a Diels-Alder reaction, an amide condensation reaction, an amide bonding reaction.

20. An artificial enzyme which exhibits enzyme activity to a target reaction and is produced by a method for producing an artificial enzyme, wherein the method for producing an artificial enzyme comprises the steps of:
selecting an artificial enzyme precursor which comprises an oligonucleotide sequence containing modified nucleosides prepared by introducing a substituent into each nucleoside and at least one of the modified nucleoside capable of reacting with a raw substance of a target reaction which the artificial enzyme catalyzes; and
producing the artificial enzyme which is capable of catalyzing the target reaction and comprises the oligonucleotide sequence in which the modified nucleoside capable of reacting with the raw substance of the artificial enzyme precursor is substituted with a non-reactive modified nucleoside which is non-reactive with the raw substance of the target reaction.


SEQUENCE LISTING (1 sequence)

SEQ ID NO: 1 - DNA, 14 bp, Artificial Sequence
  Description of Artificial SequenceDNA for manufacturing a derivative which may bond to human albumin
atgctctagcccct